(19) United States
(12) Patent Application Publication
(10) Pub. No.: US 2023/0190154 A1
(43) Pub. Date: Jun. 22, 2023

(54) SENSOR SYSTEM

(71) Applicant: Japan Display Inc., Tokyo (JP)

(72) Inventors: Chiehan CHIEN, Tokyo (JP); Masashi TAKAHATA, Tokyo (JP); Gang SHAO, Tokyo (JP)

(21) Appl. No.: 18/080,948

(22) Filed: Dec. 14, 2022

(30) Foreign Application Priority Data
Dec. 16, 2021 (JP) 2021-204034

(51) Int. Cl. A61B 5/1455; G01C 5/00; A61B 5/00

(52) U.S. Cl. CPC A61B 5/14552; G01C 5/00; A61B 5/6824; A61B 5/6826; A61B 5/0024; A61B 2560/0257

(57) ABSTRACT

According to an aspect, a sensor system includes: a first device including a first controller and a first communicator; and a plurality of second devices each including an altitude sensor, a blood oxygen saturation level sensor, a second controller, and a second communicator. The second controller of the second device is configured to transmit altitude information measured by the altitude sensor and biometric information measured by the blood oxygen saturation level sensor to the first device through the second communicator. The first device has determination information comprising determination thresholds for a blood oxygen saturation level corresponding to different altitudes. The first controller is configured to warn that the blood oxygen saturation level is lower than the determination threshold corresponding to the altitude information based on the altitude information and the biometric information received from each of the second devices through the first communicator and the determination information.

### CROSS-REFERENCE TO RELATED APPLICATION

This application claims the benefit of priority from Japanese Patent Application No. 2021-204034 filed on Dec. 16, 2021, the entire contents of which are incorporated herein by reference.

### BACKGROUND

### 1. Technical Field

What is disclosed herein relates to a sensor system.

### 2. Description of the Related Art

Detection devices having a light source and a sensor have been developed in recent years to detect a vascular pattern of, for example, veins located in a finger or a thumb, a wrist, or a foot. In a detection device of Japanese Translation of PCT International Application Publication Laid-open No. 2020-529695, the light source and the sensor are arranged so as to sandwitch an object to be detected. In such a detection device, light is emitted from the light source to the skin, and enters the body. The light then passes through the blood, muscular tissues, and the like inside the body and goes to the outside of the body to be received by the sensor.

For example, when the oxygen level in air decreases as the altitude increases and oxygen also lacks in the body during mountaineering, altitude sickness may occur in which oxygen lacks. Therefore, a leader, a guide, or the like of a mountaineering tour desirably recognize the physical conditions of its members. However, even if the physical conditions of the members can be viewed to some extent from facial complexions or behavior of the members, the actual physical conditions are difficult to be constantly recognized. Such a problem is the problem that occurs when several people act together in, for example, an excursion or an evacuation life, as well as in the mountaineering.

For the foregoing reasons, there is a need for a sensor system capable of easily allowing a change in physical conditions of members to be recognized.

### SUMMARY

According to an aspect, a sensor system includes: a first device including a first controller and a first communicator; and a plurality of second devices each including an altitude sensor, a blood oxygen saturation level sensor, a second controller, and a second communicator. The second controller of the second device is configured to transmit altitude information measured by the altitude sensor and biometric information measured by the blood oxygen saturation level sensor to the first device through the second communicator. The first device has determination information comprising determination thresholds for a blood oxygen saturation level corresponding to different altitudes. The first controller is configured to warn that the blood oxygen saturation level is lower than the determination threshold corresponding to the altitude information based on the altitude information and the biometric information received from each of the second devices through the first communicator and the determination information.

According to an aspect, a sensor system includes a plurality of wearable devices each including a controller, an altitude sensor, a blood oxygen saturation level sensor, and a communicator. Each of the wearable devices has determination information comprising determination thresholds for a blood oxygen saturation level corresponding to different altitudes. The controller is configured to share status information among the wearable devices, the status information allowing determination of whether the blood oxygen saturation level measured by the blood oxygen saturation level sensor is lower than the determination threshold corresponding to altitude information measured by the altitude sensor.

### BRIEF DESCRIPTION OF THE DRAWINGS

FIG. 1 is a configuration diagram illustrating an exemplary system configuration of a sensor system according to a first embodiment;

FIG. 2 is a schematic view illustrating a second device illustrated in FIG. 1;

FIG. 3 is a schematic view illustrating a configuration of a detection device;

FIG. 4 is a schematic view illustrating a component arrangement example of the second device with a to-be-detected object accommodated therein as viewed from one end side of the tubular second device;

FIG. 5 is a plan view illustrating the detection device according to the first embodiment;

FIG. 6 is a block diagram illustrating a configuration example of the detection device according to the first embodiment;

FIG. 7 is a circuit diagram illustrating the detection device;

FIG. 8 is a circuit diagram illustrating a plurality of partial detection areas;

FIG. 9 is a configuration diagram illustrating an exemplary functional configuration of the second device;

FIG. 10 is a configuration diagram illustrating an exemplary functional configuration of a first device according to the first embodiment;

FIG. 11 is a graph for explaining a relation example between altitude and a blood oxygen saturation level in mountaineering;

FIG. 12 is a table illustrating exemplary determination thresholds of the first device;

FIG. 13 is a plan view illustrating a display example of status information;

FIG. 14 is a flowchart illustrating an exemplary processing procedure performed by the second device according to the first embodiment;

FIG. 15 is a flowchart illustrating an exemplary processing procedure performed by the first device according to the first embodiment;

FIG. 16 is a configuration diagram illustrating an exemplary functional configuration of a first device according to a modification of the first embodiment;

FIG. 17 is a flowchart illustrating an exemplary processing procedure performed by the first device according to the modification of the first embodiment;

FIG. 18 is a configuration diagram illustrating an exemplary system configuration of a sensor system according to a second embodiment;

FIG. 19 is a configuration diagram illustrating an exemplary functional configuration of a third device according to the second embodiment; and

FIG. 20 is a flowchart illustrating an exemplary processing procedure performed by the third device according to the second embodiment.

### DETAILED DESCRIPTION

The following describes modes (embodiments) for carrying out the present invention in detail with reference to the drawings. The present invention is not limited to the description of the embodiments given below. Components described below include those easily conceivable by those skilled in the art or those substantially identical thereto. In addition, the components described below can be combined as appropriate. What is disclosed herein is merely an example, and the present invention naturally encompasses appropriate modifications easily conceivable by those skilled in the art while maintaining the gist of the invention. To further clarify the description, the drawings may schematically illustrate, for example, widths, thicknesses, and shapes of various parts as compared with actual aspects thereof. However, they are merely examples, and interpretation of the present invention is not limited thereto. The same component as that described with reference to an already mentioned drawing is denoted by the same reference numeral through the description and the drawings, and detailed description thereof will not be repeated where appropriate in some cases.

In this disclosure, when an element is described as being “on” another element, the element can be directly on the other element, or there can be one or more elements between the element and the other element.

### 1. First Embodiment

### 1-1. Sensor System

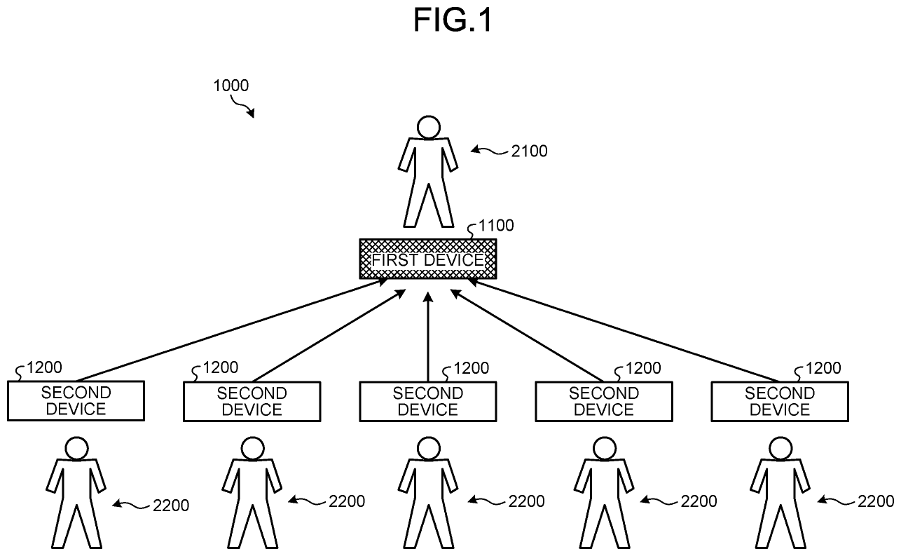

FIG. 1 is a configuration diagram illustrating an exemplary system configuration of a sensor system according to a first embodiment. As illustrated in FIG. 1, a sensor system 1000 can provide a function capable of keeping track of a change in physical conditions of a plurality of members 2200 in a group including a leader 2100 and the members 2200. Examples of the group include groups each of which includes several people for mountaineering, evacuation, an excursion, an athletic meeting, a tour, civil engineering work, or construction work. In the present embodiment, an example will be described for a case where the group is a group of several mountaineering people. In this case, the leader 2100 is the manager of the mountaineering group, and the members 2200 are climbers of the mountaineering group.

The sensor system 1000 includes a first device 1100 and a plurality of second devices 1200. Examples of the first device 1100 include a smartphone, a tablet terminal, a personal computer, and a smartwatch that allow the leader 2100 to communicate. The second devices 1200 are wearable devices that can be individually carried by the members 2200. The first device 1100 and the second devices 1200 are configured to be communicable with one another through a network or directly without a network interposed therebetween. In the present embodiment, the sensor system 1000 will be described as a case where the first device 1100 is a master device and the second devices 1200 are slave devices, but the present disclosure is not limited to this case.

In the present disclosure, the sensor system 1000 allows all the members 2200 to carry the second devices 1200, uses the second devices 1200 to measure information on living bodies, such as blood oxygen levels of the members 2200, and sequentially transmits the measurement results to the first device 1100 of the leader 2100. The sensor system 1000 can provide a function to cause the first device 1100 to monitor the data acquired from the second devices 1200, and assist the leader 2100 to quickly respond to a change in physical conditions of the members 2200.

### 1-2. Second Device

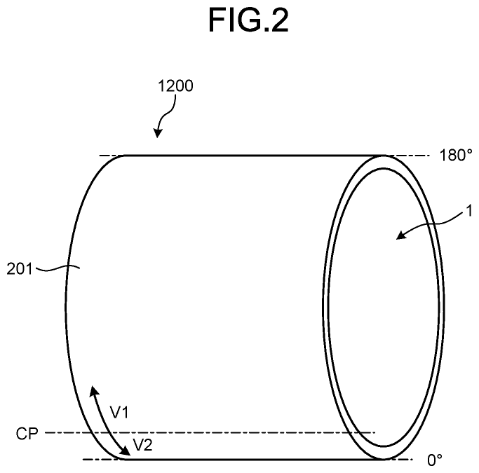

FIG. 2 is a schematic view illustrating one of the second devices 1200 according to the embodiment. The second device 1200 includes a tubular housing 201 and a detection device 1 provided in the housing 201. The housing 201 is a tubular member of, for example, a resin, a fabric, a metal, an alloy, or a ceramic and is intended to be used as a finger ring or a wristband for a person. Since the second device 1200 is intended to be used as a finger ring or a wristband, the members 2200 can easily carry the second devices 1200, which can expand the range of application of the system. In the following description, the housing 201 is assumed to be a rigid cylindrical synthetic resin intended to be used as a finger ring.

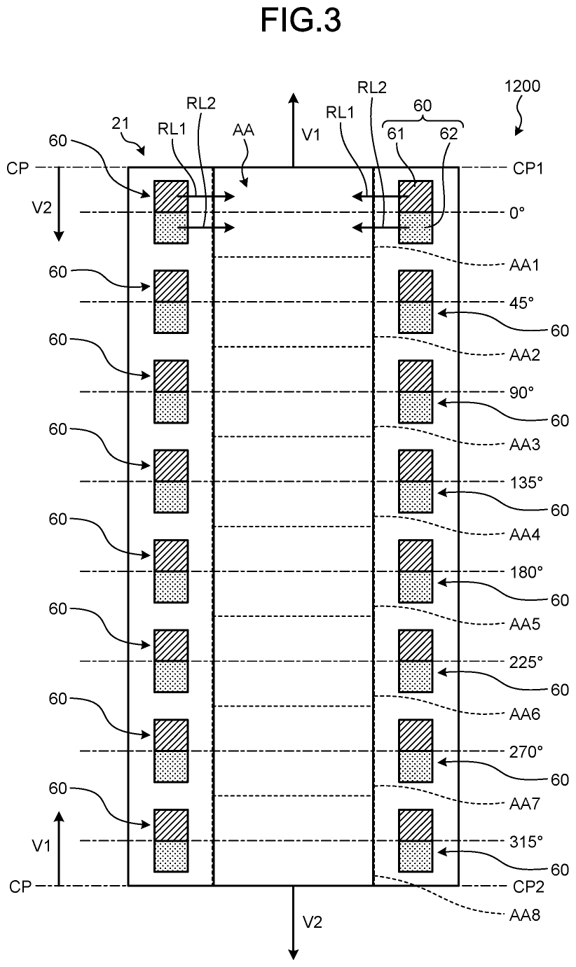

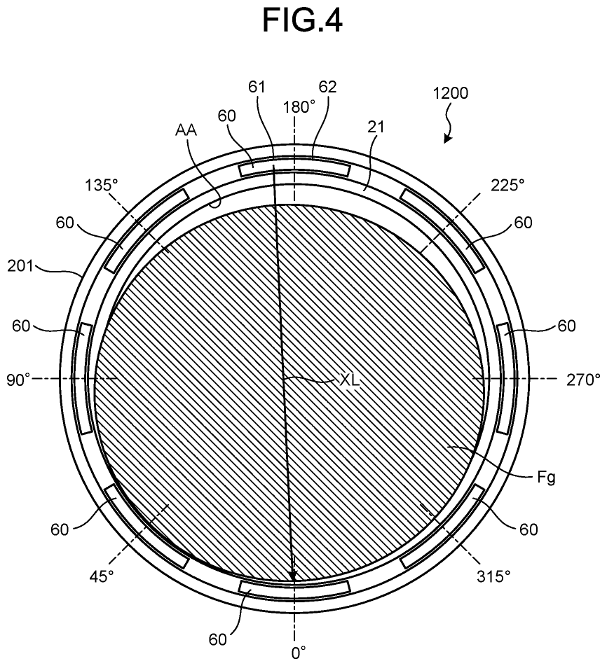

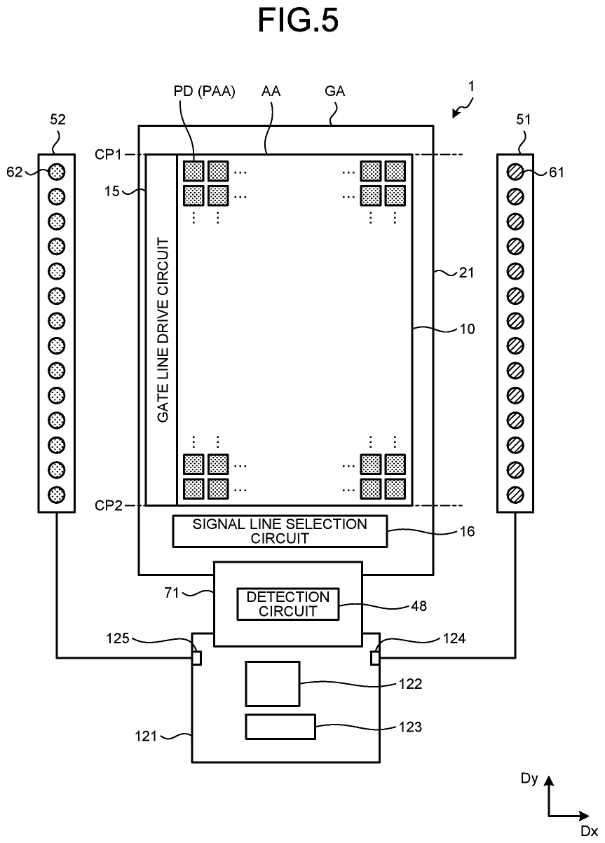

FIG. 3 is a schematic view illustrating a configuration of the detection device 1. FIG. 4 is a schematic view illustrating a component arrangement example of the second device 1200 with a finger Fg accommodated therein as viewed from one end side of the tubular second device 1200. The detection device 1 illustrated in FIGS. 3 and 4 includes a sensor substrate 21, a sensor 10 (refer to FIG. 5) including photodiodes PD provided in a detection area AA to be described later, and a plurality of light sources 60 arranged corresponding to the arrangement of the sensor 10.

As illustrated in FIG. 4, the detection area AA is provided on the inner peripheral surface of the tube of the second device 1200. The detection area AA is in contact with an object (for example, the finger Fg illustrated in FIG. 4) accommodated in the second device 1200. More specifically, the detection area AA is provided such that a coupling portion CP1 on one end side of the detection area AA illustrated in FIG. 3 is coupled to a coupling portion CP2 on the other end side of the detection area AA. With this configuration, the detection area AA forms a ring so as to continue through 360° along a ring formed by the inner peripheral surface of the second device 1200 illustrated in FIG. 2.

The description with reference to FIGS. 2 and 3 assumes that the coupling portions CP1 and CP2 illustrated in FIG. 3 are provided to contact each other in the position of a coupling portion CP illustrated in FIG. 2 so that the detection area AA forms the ring. A first direction V1 denotes a direction from the coupling portion CP illustrated in FIG. 2 toward one side along the ring formed by the inner peripheral surface of the tube of the second device 1200, and a second direction V2 denotes a direction toward the one side along the ring. In FIGS. 2, 3, and 4, a position at 0° and a position at 180° in the ring are defined to distinguish each portion of the ring formed by the detection area AA. FIGS. 3 and 4 further indicate positions at 45°, 90°, 135°, 225°, 270°, and 315° separated at intervals of 45° between the position at 0° and the position at 180°.

In FIG. 3, long dashed short dashed lines indicate the positions at 0°, 45°, 90°, 135°, 180°, 225°, 270°, and 315° from the coupling portion CP along the second direction V2. In FIG. 3, partial areas AA1, AA2, AA3, AA4, AA5, AA6, AA7, and AA8 of the detection area AA are indicated as rectangular dashed line areas. The partial areas are respectively centered in the positions at 0°, 45°, 90°, 135°, 180°, 225°, 270°, and 315°. The partial area AA1 is an area covering a range of ±22.5° centered at 0°. The partial area AA2 is an area covering a range of ±22.5° centered at 45°. The partial area AA3 is an area covering a range of ±22.5° centered at 90°. The partial area AA4 is an area covering a range of ±22.5° centered at 135°. The partial area AA5 is an area covering a range of ±22.5° centered at 180°. The partial area AA6 is an area covering a range of ±22.5° centered at 225°. The partial area AA7 is an area covering a range of ±22.5° centered at 270°. The partial area AA8 is an area covering a range of ±22.5° centered at 315°. As a base member of the sensor 10 (refer to FIG. 5) that forms the detection area AA serving as the partial areas AA1, AA2, AA3, AA4, AA5, AA6, AA7, and AA8, the sensor substrate 21 is provided so as to form a ring along the inner peripheral surface of the housing 201, as illustrated in FIG. 4.

As illustrated in FIG. 3, each of the partial areas AA1, AA2, AA3, AA4, AA5, AA6, AA7, and AA8 is provided with two of the light sources 60, and two light sources 60 face each other with a corresponding one partial area interposed therebetween. One of the two light sources 60 is located at one end side of the tube formed by the second device 1200 with respect to the detection area AA. The other of the two light sources 60 is located at the other end side of the tube formed by the second device 1200 with respect to detection area AA. Thus, as illustrated in FIG. 4, when the second device 1200 is viewed from the one end side, eight light sources 60 are arranged along the ring formed by the inner peripheral surface of the second device 1200. The light sources 60 included in the eight light sources 60 are arranged at intervals of 45°. Although not illustrated in the drawings, when the second device 1200 is viewed from the other end side, eight light sources 60 are arranged along the ring formed by the inner peripheral surface of the second device 1200, in the same manner.

Each of the light sources 60 includes a first light source 61 and a second light source 62. Thus, each of the partial areas AA1, AA2, AA3, AA4, AA5, AA6, AA7, and AA8 is provided with two of the first light sources 61, and the two first light sources 61 face each other with a corresponding one partial area interposed therebetween. Also, each of the partial areas AA1, AA2, AA3, AA4, AA5, AA6, AA7, and AA8 is provided with two of the second light sources 62, and the two second light sources 62 face each other with a corresponding one partial area interposed therebetween. In the ring formed by the inner peripheral surface of the second device 1200, the first light source 61 is located on the first direction V1 side of the second light source 62. In the ring formed by the inner peripheral surface of the second device 1200, the second light source 62 is located on the second direction V2 side of the first light source 61. As illustrated in FIG. 3, a boundary line between the first and the second light sources 61 and 62 included in one of the light sources 60 overlaps one of the long dashed short dashed lines indicating 0°, 45°, 90°, 135°, 180°, 225°, 270°, and 315°. Light XL illustrated in FIG. 4 is light (such as infrared light) that has been emitted from the first light source 61 and transmitted through the finger Fg.

The following describes an exemplary configuration of the detection device 1. FIG. 5 is a plan view illustrating the detection device 1 according to the first embodiment. As illustrated in FIG. 5, a detection device 1 includes the sensor substrate 21, the sensor 10, a gate line drive circuit 15, a signal line selection circuit 16, a detection circuit 48, a control circuit 122, a power supply circuit 123, a first light source base member 51, a second light source base member 52, the first light source 61, and the second light source 62.

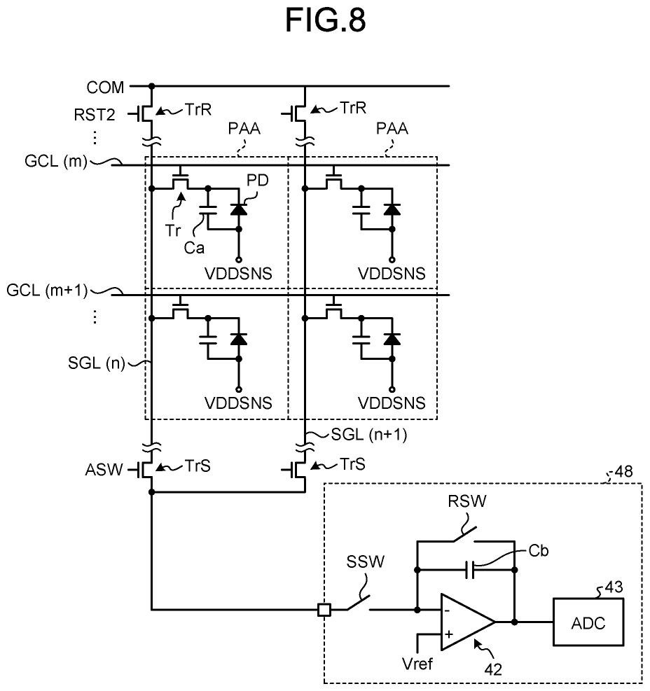

The sensor substrate 21 is electrically coupled to a control board 121 through a flexible printed circuit board 71. The flexible printed circuit board 71 is provided with the detection circuit 48. The control board 121 is provided with the control circuit 122 and the power supply circuit 123. The control circuit 122 is, for example, a field-programmable gate array (FPGA). The control circuit 122 supplies control signals to the sensor 10, the gate line drive circuit 15, and the signal line selection circuit 16 to control a detection operation of the sensor 10. The control circuit 122 supplies control signals to the first and the second light sources 61 and 62 to control lighting and non-lighting of the first and the second light sources 61 and 62. The power supply circuit 123 supplies voltage signals including, for example, a sensor power supply signal (sensor power supply voltage) VDDSNS (refer to FIG. 8) to the sensor 10, the gate line drive circuit 15, and the signal line selection circuit 16. The power supply circuit 123 supplies a power supply voltage to the first and the second light sources 61 and 62.

The sensor substrate 21 has the detection area AA and a peripheral area GA. The detection area AA is an area provided with the photodiodes PD included in the sensor 10. The peripheral area GA is an area between the outer perimeter of the detection area AA and the edges of the sensor substrate 21 and is an area not overlapping the photodiodes PD.

A position overlapping one side of the four sides of the detection area AA that form a boundary between the rectangular detection area AA and the peripheral area GA serves as the coupling portion CP1 illustrated in FIG. 3. A position overlapping another side of the four sides of the detection area AA that faces the one side with the detection area AA interposed therebetween serves as the coupling portion CP2 illustrated in FIG. 3.

The gate line drive circuit 15 and the signal line selection circuit 16 are provided in the peripheral area GA. Specifically, the gate line drive circuit 15 is provided in an area extending along a second direction Dy in the peripheral area GA. The signal line selection circuit 16 is provided in an area extending along a first direction Dx in the peripheral area GA and is provided between the sensor 10 and the detection circuit 48.

The first direction Dx is one direction in a plane parallel to the sensor substrate 21. The second direction Dy is one direction in the plane parallel to the sensor substrate 21 and is a direction orthogonal to the first direction Dx. The second direction Dy may non-orthogonally intersect the first direction Dx. A third direction Dz is a direction orthogonal to the first direction Dx and the second direction Dy and is a direction normal to the sensor substrate 21.

The first light sources 61 are provided on the first light source base member 51 and are arranged along the second direction Dy. The second light sources 62 are provided on the second light source base member 52 and are arranged along the second direction Dy. The first light source base member 51 and the second light source base member 52 are electrically coupled to the control circuit 122 and the power supply circuit 123 through respective terminals 124 and 125 provided on the control board 121.

For example, inorganic light-emitting diodes (LEDs) or organic electroluminescent (EL) diodes (organic light-emitting diodes (OLEDs)) are used as the first and the second light sources 61 and 62. The first and the second light sources 61 and 62 respectively emit first light and second light having different wavelengths. In the present embodiment, the first light source 61 emits infrared light having a wavelength of 880 nm. The second light source 62 emits red light having a wavelength of 665 nm. During detection, the first light source 61 and the second light source 62 are alternately lit up. Thus, the photodiodes PD alternately receive reflected light L2 of the infrared light and the red light.

The reflected light of the infrared light contains information for detecting a vascular pattern. Red blood cells contained in blood contain hemoglobin. The infrared light emitted from the first light source 61 can be easily absorbed by hemoglobin. In other words, the coefficient of absorption of infrared light by hemoglobin is higher than that of the other portions in the body. Therefore, the vascular pattern of, for example, veins can be detected by reading the amount of light received by the photodiodes PD, and identifying locations where the amount of the received reflected light L2 of the infrared light is relatively small.

The reflected light of the infrared light and the red light contains information for measuring an oxygen saturation level in blood (hereinafter, called “blood oxygen saturation level (SpO₂)”). The blood oxygen saturation level (SpO₂) is the ratio of the amount of oxygen actually bound to hemoglobin to the total amount of oxygen under the assumption that the oxygen is bound to all the hemoglobin in the blood.

The infrared light can be easily absorbed by hemoglobin. Increasing the amount of hemoglobin increases the amount of absorbed infrared light, and reduces the amount of light received by the photodiodes PD. That is, the total amount of hemoglobin is determined from the amount of the received reflected light L2 of the infrared light.

The hemoglobin has a dark red color when not bound to oxygen and has a bright red color when bound to oxygen. Therefore, the absorption coefficient of the hemoglobin for absorbing the red light differs between when the hemoglobin is bound to oxygen and when the hemoglobin is not bound to oxygen. As a result, the amount of the reflected light of the red light increases as the hemoglobin bound to oxygen increases in the blood. In contrast, the amount of the reflected light of the red light decreases as the hemoglobin not bound to oxygen increases in the blood. Thus, the amount of the hemoglobin bound to oxygen is relatively determined based on the amount of the received reflected light of the red light.

Then, by comparing the determined total amount of the hemoglobin with the amount of the hemoglobin bound to oxygen, the ratio of the amount of oxygen actually bound to the hemoglobin (blood oxygen saturation level (SpO₂)) can be determined. Thus, the detection device 1 includes the first and the second light sources 61 and 62, and therefore, can detect information on a living body in the finger Fg or the like by performing the detection based on the first light and the detection based on the second light. The detection device 1 can supply the detected information on the living body including, for example, the blood oxygen saturation level and pulsation to the control board 121 through the flexible printed circuit board 71.

In the present disclosure, the wavelengths of the light emitted from the first and the second light sources 61 and 62 are not limited to the wavelengths exemplified above. The first light source 61 only needs to emit the infrared light having a wavelength of from 800 nm to 1000 nm. The second light source 62 only needs to emit the red light having a wavelength of from 600 nm to 800 nm.

The arrangement of the first and the second light sources 61 and 62 illustrated in FIG. 5 is merely an example and may be changed as appropriate. For example, the first and the second light sources 61 and 62 may be arranged on each of the first and the second light source base members 51 and 52. In this case, a group including the first light sources 61 and a group including the second light sources 62 may be arranged in the second direction Dy, or the first and the second light sources 61 and 62 may be arranged alternately in the second direction Dy. The first and the second light sources 61 and 62 may be provided on one light source base member, or three or more light source base members.

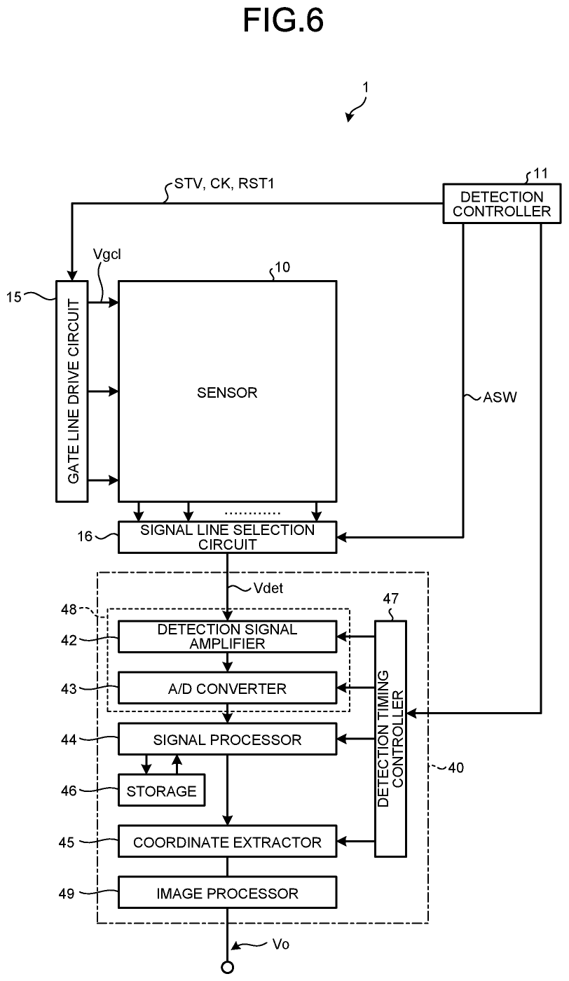

FIG. 6 is a block diagram illustrating a configuration example of the detection device 1 according to the present embodiment. As illustrated in FIG. 6, the detection device 1 further includes a detection controller (detection control circuitry) 11 and a detector (detection circuitry) 40. The control circuit 122 includes one, some, or all functions of the detection controller 11. The control circuit 122 also includes one, some, or all functions of the detector 40 except those of the detection circuit 48.

The sensor 10 is an optical sensor that includes the photodiodes PD serving as photoelectric conversion elements. Each of the photodiodes PD included in the sensor 10 outputs an electrical signal corresponding to light received by the photodiode PD as a detection signal Vdet to the signal line selection circuit 16. The sensor 10 performs the detection in response to a gate drive signal Vgcl supplied from the gate line drive circuit 15.

The detection controller 11 is a circuit that supplies respective control signals to the gate line drive circuit 15, the signal line selection circuit 16, and the detector 40 to control operations of these components. The detection controller 11 supplies various control signals including, for example, a start signal STV, a clock signal CK, and a reset signal RST1 to the gate line drive circuit 15. The detection controller 11 also supplies various control signals including, for example, a selection signal ASW to the signal line selection circuit 16. The detection controller 11 supplies various control signals to the first and the second light sources 61 and 62 to control the lighting and non-lighting of the first and second light sources 61 and 62.

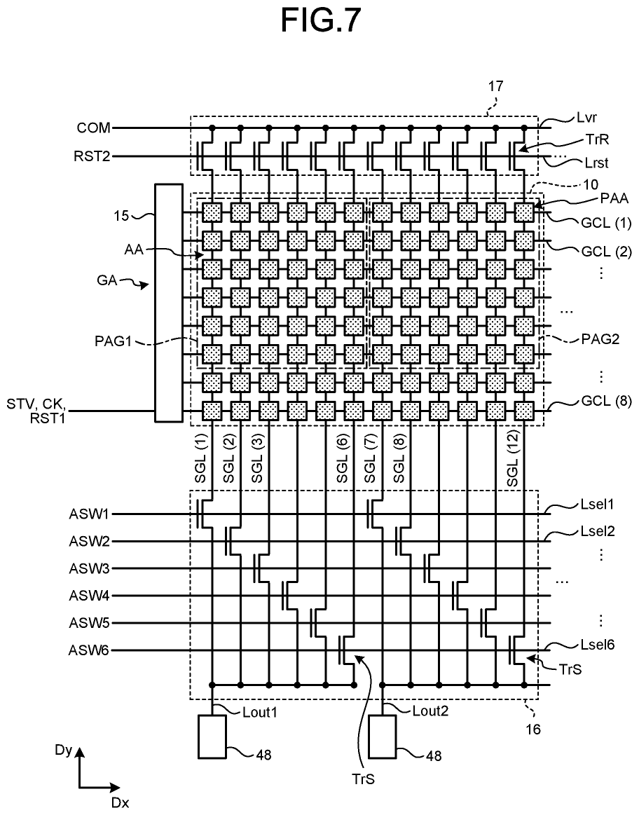

The gate line drive circuit 15 is a circuit that drives a plurality of gate lines GCL (refer to FIG. 7) based on the various control signals. The gate line drive circuit 15 sequentially or simultaneously selects the gate lines GCL and supplies the gate drive signals Vgcl to the selected gate lines GCL. By this operation, the gate line drive circuit 15 selects the photodiodes PD coupled to the gate lines GCL.

The signal line selection circuit 16 is a switch circuit that sequentially or simultaneously selects a plurality of signal lines SGL (refer to FIG. 7). The signal line selection circuit 16 is, for example, a multiplexer. The signal line selection circuit 16 couples the selected signal lines SGL to the detection circuit 48 based on the selection signal ASW supplied from the detection controller 11. By this operation, the signal line selection circuit 16 outputs the detection signals Vdet of the photodiodes PD to the detector 40.

The detector 40 includes the detection circuit 48, a signal processor (signal processing circuit) 44, a coordinate extractor (coordinate extraction circuit) 45, a storage (storage circuit) 46, a detection timing controller (timing control circuit) 47, and an image processor (image processing circuit) 49. Based on a control signal supplied from the detection controller 11, the detection timing controller 47 controls the detection circuit 48, the signal processor 44, the coordinate extractor 45, and the image processor 49 such that they operate in synchronization with one another.

The detection circuit 48 is, for example, an analog front-end (AFE) circuit. The detection circuit 48 is a signal processing circuit having functions of at least a detection signal amplifier 42 and an analog-to-digital (A/D) converter 43. The detection signal amplifier 42 amplifies the detection signals Vdet. The A/D converter 43 converts analog signals output from the detection signal amplifier 42 into digital signals.

The signal processor 44 is a logic circuit that detects a predetermined physical quantity received by the sensor 10 based on an output signal of the detection circuit 48. The signal processor 44 can detect asperities on a surface of the finger Fg or a palm based on the signals from the detection circuit 48 when the finger Fg is in contact with or in proximity to a detection surface. The signal processor 44 can also detect the information on the living body based on the signals from the detection circuit 48. Examples of the information on the living body include the pulsation and the blood oxygen saturation level of the finger Fg.

The signal processor 44 may also perform processing of acquiring the detection signals Vdet (information on the living body) simultaneously detected by the photodiodes PD, and averaging the detection signals Vdet. In this case, the detector 40 can perform stable detection by reducing measurement errors caused by noise and/or relative positional misalignment between an object to be detected, such as the finger Fg, and the sensor 10.

The storage 46 temporarily stores therein signals calculated by the signal processor 44. The storage 46 may be, for example, a random-access memory (RAM) or a register circuit.

The coordinate extractor 45 is a logic circuit that obtains detected coordinates of the asperities on the surface of the finger or the like when the contact or the proximity of the finger is detected by the signal processor 44. The coordinate extractor 45 is also a logic circuit that obtains detected coordinates of blood vessels of the finger Fg or the palm. The image processor 49 combines the detection signals Vdet output from the respective photodiodes PD of the sensor 10 to generate two-dimensional information representing the shape of the asperities on the surface of the finger Fg or the like and two-dimensional information representing the shape of the blood vessels of the finger Fg or the palm. The coordinate extractor 45 may output the detection signals Vdet as sensor outputs Vo instead of calculating the detected coordinates. There may be a case where the detector 40 does not include the coordinate extractor 45 and the image processor 49.

The following describes a circuit configuration example of the detection device 1. FIG. 7 is a circuit diagram illustrating the detection device 1. FIG. 8 is a circuit diagram illustrating a plurality of partial detection areas. FIG. 8 also illustrates a circuit configuration of the detection circuit 48.

As illustrated in FIG. 7, the sensor 10 has a plurality of partial detection areas PAA arranged in a matrix having a row-column configuration. Each of the partial detection areas PAA is provided with the photodiode PD.

The gate lines GCL extend in the first direction Dx, and are coupled to the partial detection areas PAA arranged in the first direction Dx. A plurality of gate lines GCL(1), GCL(2), . . . , GCL(8) are arranged in the second direction Dy and are each coupled to the gate line drive circuit 15. In the following description, the gate lines GCL(1), GCL(2), . . . , GCL(8) will each be simply referred to as the gate line GCL when they need not be distinguished from one another. For ease of understanding of the description, FIG. 7 illustrates eight of the gate lines GCL. However, this is merely an example, and M gate lines GCL (where M is eight or larger, and is, for example, 256) may be arranged.

The signal lines SGL extend in the second direction Dy and are each coupled to the photodiodes PD of the partial detection areas PAA arranged in the second direction Dy. A plurality of signal lines SGL(1), SGL(2), . . . , SGL(12) are arranged in the first direction Dx and are each coupled to the signal line selection circuit 16 and a reset circuit 17. In the following description, the signal lines SGL(1), SGL(2), . . . , SGL(12) will each be simply referred to as the signal line SGL when they need not be distinguished from one another.

For ease of understanding of the description, 12 of the signal lines SGL are illustrated. However, this is merely an example, and N signal lines SGL (where N is 12 or larger, and is, for example, 252) may be arranged. In FIG. 7, the sensor 10 is provided between the signal line selection circuit 16 and the reset circuit 17. The present disclosure is not limited thereto. The signal line selection circuit 16 and the reset circuit 17 may be coupled to ends of the signal lines SGL on the same side.

The gate line drive circuit 15 receives the various control signals such as the start signal STV, the clock signal CK, and the reset signal RST1 from the control circuit 122 (refer to FIG. 5). The gate line drive circuit 15 sequentially selects the gate lines GCL(1), GCL(2), . . . , GCL(8) in a time-division manner based on the various control signals. The gate line drive circuit 15 supplies the gate drive signal Vgcl to the selected one of the gate lines GCL. This operation supplies the gate drive signal Vgcl to a plurality of first switching elements Tr coupled to the gate line GCL, and the partial detection areas PAA arranged in the first direction Dx are selected as detection targets.

The gate line drive circuit 15 may perform different driving for each of detection modes including the detection of the fingerprint and the detections of different items of the information on the living body (such as the pulsation and the blood oxygen saturation level). For example, the gate line drive circuit 15 may drive more than one of the gate lines GCL collectively.

Specifically, the gate line drive circuit 15 simultaneously selects a predetermined number of the gate lines GCL from among the gate lines GCL(1), GCL(2), . . . , GCL(8) based on the control signals. For example, the gate line drive circuit 15 simultaneously selects six gate lines GCL(1) to GCL(6) and supplies thereto the gate drive signals Vgcl. The gate line drive circuit 15 supplies the gate drive signals Vgcl through the selected six gate lines GCL to the first switching elements Tr. By this operation, detection area groups PAG1 and PAG2 each including more than one of the partial detection areas PAA arranged in the first direction Dx and the second direction Dy are selected as the detection targets. The gate line drive circuit 15 drives the predetermined number of the gate lines GCL collectively, and sequentially supplies the gate drive signals Vgcl to the gate lines GCL in units of the predetermined number of the gate lines GCL.

The signal line selection circuit 16 includes a plurality of selection signal lines Lsel, a plurality of output signal lines Lout, and a plurality of third switching elements TrS. The third switching elements TrS are provided corresponding to the signal lines SGL. Six signal lines SGL(1), SGL(2), . . . , SGL(6) are coupled to a common output signal line Lout1. Six signal lines SGL(7), SGL(8), . . . , SGL(12) are coupled to a common output signal line Lout2. The output signal lines Lout1 and Lout2 are each coupled to the detection circuit 48.

The signal lines SGL(1), SGL(2), . . . , SGL(6) are grouped into a first signal line block, and the signal lines SGL(7), SGL(8), . . . , SGL(12) are grouped into a second signal line block. The selection signal lines Lsel are coupled to the gates of the respective third switching elements TrS included in one of the signal line blocks. One of the selection signal lines Lsel is coupled to the gates of the third switching elements TrS in the signal line blocks.

Specifically, selection signal lines Lsel1, Lsel2, . . . , Lsel6 are coupled to the third switching elements TrS corresponding to the signal lines SGL(1), SGL(2), . , SGL(6), respectively. The selection signal line Lsel1 is coupled to one of the third switching elements TrS corresponding to the signal line SGL(1) and one of the third switching elements TrS corresponding to the signal line SGL(7). The selection signal line Lsel2 is coupled to one of the third switching elements TrS corresponding to the signal line SGL(2) and one of the third switching elements TrS corresponding to the signal line SGL(8).

The control circuit 122 (refer to FIG. 5) sequentially supplies the selection signal ASW to the selection signal lines Lsel. This operation causes the signal line selection circuit 16 to operate the third switching elements TrS to sequentially select the signal lines SGL in one of the signal line blocks in a time-division manner. The signal line selection circuit 16 selects the signal lines SGL one by one in each of the signal line blocks. With the above-described configuration, the detection device 1 can reduce the number of integrated circuits (ICs) including the detection circuit 48 or the number of terminals of the ICs.

The signal line selection circuit 16 may couple more than one of the signal lines SGL collectively to the detection circuit 48. Specifically, the control circuit 122 (refer to FIG. 5) simultaneously supplies the selection signal ASW to the selection signal lines Lsel. This operation causes the signal line selection circuit 16 to operate the third switching elements TrS to select more than one of the signal lines SGL (for example, six of the signal lines SGL) in one of the signal line blocks, and couple the signal lines SGL to the detection circuit 48. As a result, the signals detected in each of the detection area groups PAG1 and PAG2 are output to the detection circuit 48. In this case, the signals from the partial detection areas PAA (photodiodes PD) included in each of the detection area groups PAG1 and PAG2 are integrated together and output to the detection circuit 48.

By the operations of the gate line drive circuit 15 and the signal line selection circuit 16, the detection is performed for each of the detection area groups PAG1 and PAG2. As a result, the intensity of the first detection signal Vdet obtained by a single detection operation increases, so that the sensor sensitivity can be improved. The time required for the detection can also be reduced. As a result, the detection device 1 can repeatedly perform the detection in a short time, and thus, can improve the signal-to-noise ratio (S/N), and can also accurately detect time-varying changes in the information on the living body, such as a pulse wave.

As illustrated in FIG. 7, the reset circuit 17 includes a reference signal line Lvr, a reset signal line Lrst, and fourth switching elements TrR. The fourth switching elements TrR are provided corresponding to the signal lines SGL. The reference signal line Lvr is coupled to either the sources or the drains of the fourth switching elements TrR. The reset signal line Lrst is coupled to the gates of the fourth switching elements TrR.

The control circuit 122 supplies a reset signal RST2 to the reset signal line Lrst. This operation turns on the fourth switching elements TrR to electrically couple the signal lines SGL to the reference signal line Lvr. The power supply circuit 123 supplies a reference signal COM to the reference signal line Lvr. This operation supplies the reference signal COM to a capacitive element Ca (refer to FIG. 8) included in each of the partial detection areas PAA.

As illustrated in FIG. 8, each of the partial detection areas PAA includes the photodiode PD, the capacitive element Ca, and the first switching element Tr. FIG. 8 illustrates two gate lines GCL(m) and GCL(m+1) arranged in the second direction Dy among the gate lines GCL. FIG. 8 also illustrates two signal lines SGL(n) and SGL(n+1) arranged in the first direction Dx among the signal lines SGL. The partial detection area PAA is an area surrounded by the gate lines GCL and the signal lines SGL. The first switching elements Tr are provided so as to correspond to the photodiodes PD. The first switching element Tr is formed of a thin-film transistor, and in this example, formed of an n-channel metal oxide semiconductor (MOS) thin-film transistor (TFT).

The gates of the first switching elements Tr belonging to the partial detection areas PAA arranged in the first direction Dx are coupled to the gate line GCL. The sources of the first switching elements Tr belonging to the partial detection areas PAA arranged in the second direction Dy are coupled to the signal line SGL. The drain of the first switching element Tr is coupled to the cathode of the photodiode PD and the capacitive element Ca.

The anode of the photodiode PD is supplied with the sensor power supply signal (sensor power supply voltage) VDDSNS from the power supply circuit 123. The signal line SGL and the capacitive element Ca are supplied with the reference signal COM that serves as an initial potential of the signal line SGL and the capacitive element Ca from the power supply circuit 123.

When the partial detection area PAA is irradiated with light, a current corresponding to the amount of the light flows through the photodiode PD. As a result, an electric charge is stored in the capacitive element Ca. After the first switching element Tr is turned on, a current corresponding to the electric charge stored in the capacitive element Ca flows through the signal line SGL. The signal line SGL is coupled to the detection circuit 48 through one of the third switching elements TrS of the signal line selection circuit 16. Thus, the detection device 1 can detect a signal corresponding to the amount of the light received by the photodiode (photodiodes) PD in each of the partial detection areas PAA or each of the detection area groups PAG1 and PAG2.

During a read period, a switch SSW of the detection circuit 48 is turned on, and the detection circuit 48 is coupled to the signal lines SGL. The detection signal amplifier 42 of the detection circuit 48 converts a current supplied from the signal lines SGL into a voltage corresponding to the value of the current, and amplifies the result. A non-inverting input terminal (+) of the detection signal amplifier 42 receives a reference voltage Vref having a fixed potential, and an inverting input terminal (−) of the detection signal amplifier 42 is coupled to the signal lines SGL. In the present embodiment, the same signal as the reference signal COM is received as the reference voltage Vref. The detection signal amplifier 42 includes a capacitive element Cb and a reset switch RSW. During a reset period, the reset switch RSW is turned on to reset the electric charge of the capacitive element Cb.

With the above-described configuration, the detection device 1, which includes the photodiodes PD, can detect the information on the living body, such as the blood oxygen saturation level and the pulsation of the target object and supply biometric information including the detected information to the outside of the unit.

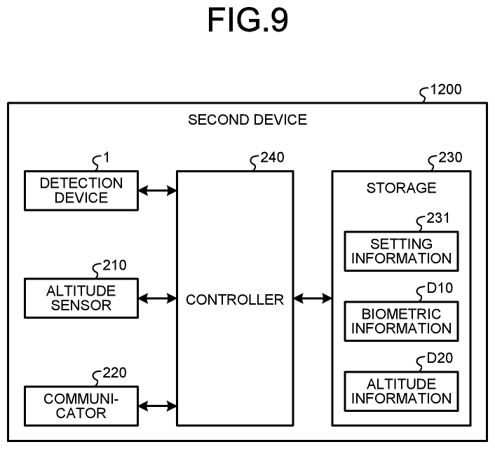

The following describes a functional configuration of the second device 1200. FIG. 9 is a configuration diagram illustrating an example of the functional configuration of the second device 1200. As illustrated in FIG. 9, the second device 1200 includes the detection device 1 described above, an altitude sensor 210, a communicator (communication circuit) 220, a storage (storage circuit) 230, and a controller (control circuit 240. The controller 240 is electrically coupled to the detection device 1, the altitude sensor 210, the communicator 220, and the storage 230.

The detection device 1 measures biometric information D10 on a living body in the finger Fg or the like of each of the members 2200 and supplies the biometric information D10 to the controller 240. The detection device 1 supplies the biometric information D10 to the controller 240, for example, each time the biometric information D10 is measured at predetermined timing. Examples of the predetermined timing include “at a set cycle”, “at set intervals”, and “at a set time”. In the present embodiment, the detection device 1 serves as a blood oxygen saturation level sensor by supplying the biometric information D10 including the blood oxygen saturation level to the controller 240.

The altitude sensor 210 measures information on the altitude of the second device 1200. The altitude sensor 210 includes, for example, a barometric pressure sensor, a Global Positioning System (GPS) receiver, and an altimeter. The altitude sensor 210 has a function to measure the ambient barometric pressure acting on the second device 1200 and convert the barometric pressure into the altitude. The altitude sensor 210 calculates the altitude from the measured barometric pressure using a conversion computer program, a conversion table, and/or the like. The altitude sensor 210 calculates the altitude (elevation) of the current position (latitude and longitude) of the second device 1200 measured by the GPS receiver, based on the relation of the latitude and the longitude with the height indicated by map information. The map information includes, for example, information in which information on the latitude, the longitude, and the altitude (elevation) is mapped. The altitude sensor 210 supplies altitude information D20 indicating the altitude and the barometric pressure measured to the controller 240. In the present embodiment, the configuration is described where the altitude sensor 210 is not included in the detection device 1, but the altitude sensor 210 may be included in the detection device 1. If the leader 2100 is assumed to act with the members 2200, the second device 1200 may have a configuration not including the altitude sensor 210, and the first device 1100 may regard the altitude of itself as the altitudes of the members 2200.

The communicator 220 wirelessly communicates. The communicator 220 supports wireless communication standards. Examples of the communication standards include the cellular phone communication standards, such as the third generation (3G), the fourth generation (4G), and the fifth generation (5G) standards, and the short-range wireless communication standards. The communicator 220 supplies received information to the controller 240. The communicator 220 transmits various types of information requested by the controller 240 to destinations. In the present embodiment, the communicator 220 serves as a second communicator.

The storage 230 stores therein computer programs and data. The storage 230 temporarily stores therein processing results of the controller 240. The storage 230 includes storage media. The storage media include, for example, a read-only memory (ROM), a random-access memory (RAM), a memory card, an optical disc, and/or a magneto-optical disk. The storage 230 stores therein information indicating of, for example, detection results detected by the detection device 1 and the altitude sensor 210.

The storage 230 stores therein, for example, setting information 231, the biometric information D10, and the altitude information D20. The setting information 231 is various types of information set for the second device 1200. The setting information 231 includes, for example, identifying information allowing identification of, for example, the second devices 1200, the members 2200 carrying the second devices 1200, and destination information. The setting information 231 includes information allowing identification of, for example, the relation between the master and the slaves in the system and the devices belonging to the group. The biometric information D10 is information supplied by the detection device 1 and includes the information on the living body, such as the pulsation and the blood oxygen saturation level of the finger Fg or the palm. The altitude information D20 is information supplied by the altitude sensor 210 and includes information on, for example, the altitude and the barometric pressure that have been detected. The storage 230 stores therein the biometric information D10 and the altitude information D20 detected at the same time or at approximately the same time in association with each other.

The controller 240 includes, for example, a microcontrol unit (MCU) and/or a central processing unit (CPU). The controller 240 integrally controls the operation of the second device 1200. The controller 240 has, for example, a function to transmit the biometric information D10 or the like to the first device 1100 and a function to acquire the altitude information D20. Various functions of the controller 240 are performed by executing the computer programs. In the present embodiment, the controller 240 serves as a second controller.

The controller 240 stores the biometric information D10 from the detection device 1 and the altitude information D20 from the altitude sensor 210 in association with each other in the storage 230. The controller 240 transmits the biometric information D10 measured by the detection device 1 and the altitude information D20 measured by the altitude sensor 210 to the first device 1100 through the communicator 220. In the present embodiment, the controller 240 serves as the second controller that transmits the altitude information D20 measured by the altitude sensor 210 and the biometric information D10 measured by the detection device 1 (blood oxygen saturation level sensor) to the first device 1100 through the communicator 220 (second communicator). The controller 240 may transmit the biometric information D10 and the altitude information D20 to the first device 1100 at different times.

The above has described the functional configuration example of the second device 1200 according to the present embodiment. The configuration described above using FIGS. 2 to 9 is merely an example. The functional configuration of the second device 1200 according to the present embodiment is not limited to this example. The functional configuration of the second device 1200 according to the present embodiment can be flexibly modified according to specifications and operations.

### 1-3. First Device

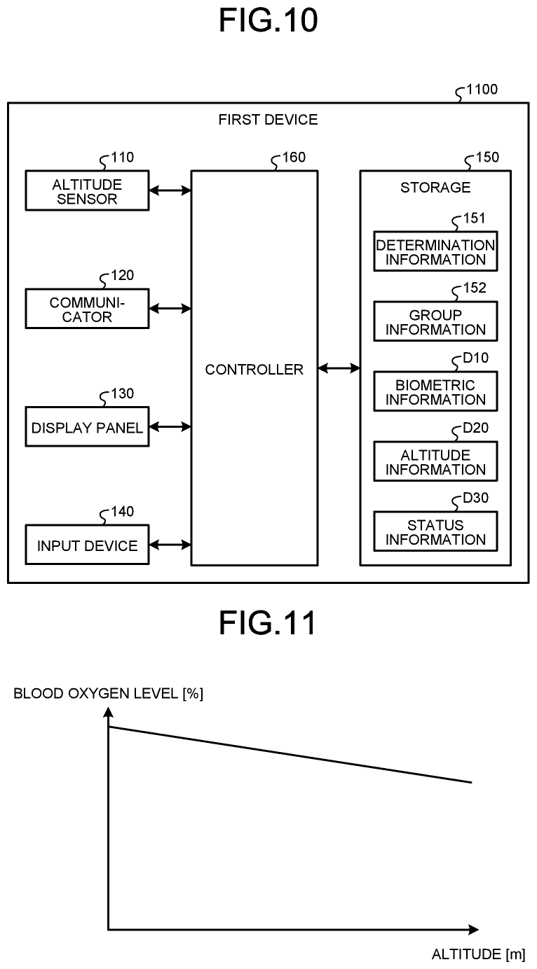

The following describes a functional configuration of the first device 1100. FIG. 10 is a configuration diagram illustrating an exemplary functional configuration of the first device 1100 according to the first embodiment. As illustrated in FIG. 10, the first device 1100 includes an altitude sensor 110, a communicator (communication circuit) 120, a display panel 130, an input device (input circuit) 140, a storage (storage circuit) 150, and a controller (control circuit) 160. The controller 160 is electrically coupled to the altitude sensor 110, the communicator 120, the display panel 130, the input device 140, and the storage 150.

The altitude sensor 110 includes, for example, a barometric pressure sensor, a GPS receiver, and an altimeter. The altitude sensor 110 has a function to measure the ambient barometric pressure acting on the first device 1100 and convert the barometric pressure into the altitude. The altitude sensor 110 calculates the altitude from the measured barometric pressure using a conversion computer program, a conversion table, and/or the like. The altitude sensor 110 calculates the altitude (elevation) of the current position (latitude and longitude) of the first device 1100 measured by the GPS receiver, based on the relation of the latitude and the longitude with the height indicated by the map information. The map information includes, for example, the information in which the information on the latitude, the longitude, and the altitude (elevation) is mapped. The altitude sensor 110 supplies the altitude information D20 indicating, for example, the altitude and the barometric pressure measured to the controller 160.

The communicator 120 wirelessly communicates. The communicator 120 supports the wireless communication standards. Examples of the communication standards include the cellular phone communication standards, such as the 3G, the 4G, and the 5G standards, and the short-range wireless communication standards. The communicator 120 supplies received information to the controller 160. The communicator 120 transmits various types of information requested by the controller 160 to destinations. In the present embodiment, the communicator 120 serves as a first communicator.

The display panel 130 has a function to display various types of information. The display panel 130 displays, for example, information received from the second device 1200 and information for assisting. The controller 160 controls display of the display panel 130. For example, a display device that displays various types of information can be used as the display panel 130. Examples of the display device include a liquid crystal display and an organic EL display.

The input device 140 has a function to detect physical input operations by a user. The input device 140 includes operation devices, such as a touchscreen and/or operation buttons. The input device 140 supplies input information indicating the detected input operations to the controller 160.

The storage 150 stores therein computer programs and data. The storage 150 temporarily stores therein processing results of the controller 160. The storage 150 includes storage media. The storage media include, for example, a ROM, a RAM, a memory card, an optical disc, and/or a magneto-optical disk. The storage 150 stores therein information indicating, for example, the measurement results measured by the altitude sensor 110.

The storage 150 stores therein, for example, determination information 151, group information 152, the biometric information D10, the altitude information D20, and status information D30. The determination information 151 includes determination thresholds for the blood oxygen saturation level corresponding to, for example, a plurality of altitudes, altitude intervals, altitude ranges. Each of the determination thresholds is a threshold for determining whether the blood oxygen saturation level is abnormal. In other words, the determination threshold is a threshold allowing determination of whether an abnormality in the blood oxygen saturation level needs to be reported as a warning. An example of the determination information 151 will be described later. The group information 152 includes information allowing identification of, for example, the members 2200 in the group managed by the leader 2100 and the second devices 1200 carried by the members 2200. For example, the group information 152 includes information indicating, for example, a table that associates the members 2200 with the second devices 1200. The group information 152 includes information allowing identification of the second devices 1200 that are set as the communicable slaves by the first device 1100 serving as the master. The second devices 1200 that communicate with the first device 1100 are the second devices 1200 carried by the members 2200 managed by the leader 2100. For example, the group information 152 includes information indicating a relation between each of the members 2200 to be managed and the second device 1200 associated with the member 2200.

The biometric information D10 is the biometric information D10 received from the second device 1200, and is associated with at least either of the member 2200 and the second device 1200. The altitude information D20 includes information supplied by the altitude sensor 110. The altitude information D20 includes information received from the second device 1200 and is associated with at least either of the member 2200 and the second device 1200. The storage 150 stores therein the biometric information D10 and the altitude information D20 in association with at least either of the member 2200 and the second device 1200. The status information D30 includes information allowing determination of whether the blood oxygen saturation level measured by each of the second devices 1200 is in a dangerous range. In the present embodiment, a case will be described where the status information D30 includes information indicating a determination result of whether the blood oxygen saturation level of each of the members 2200 is normal, cautionary, or dangerous. However, the information may, for example, indicate the member 2200 who is determined to be abnormal in the blood oxygen saturation level.

The controller 160 includes, for example, an MCU and a CPU. The controller 160 integrally controls the operation of the first device 1100. The controller 160 has, for example, a function to acquire the altitude information D20 and the biometric information D10 received from each of the second devices 1200 through the communicator 120. The controller 160 has a function to warn that the blood oxygen saturation level is lower than the determination threshold corresponding to the altitude information D20, based on the altitude information D20 and the biometric information D10 thus received and the determination information 151. The controller 160 has a function to display the status information allowing determination of whether the blood oxygen saturation level measured by each of the second devices 1200 is in the dangerous range on the display panel 130. The controller 160 has a function to manage the measurement results measured by the first device 1100 and the second devices 1200, and warn that the blood oxygen saturation level is lower than the determination threshold corresponding to the altitude information D20. Various functions of the controller 160 are performed by executing the computer programs. In the present embodiment, the controller 160 serves as a first controller.

The above has described the functional configuration example of the first device 1100 according to the present embodiment. The configuration described above using FIG. 10 is merely an example. The functional configuration of the first device 1100 according to the present embodiment is not limited to this example. The functional configuration of the first device 1100 according to the present embodiment can be flexibly modified according to specifications and operations.

### Determination Thresholds of First Device

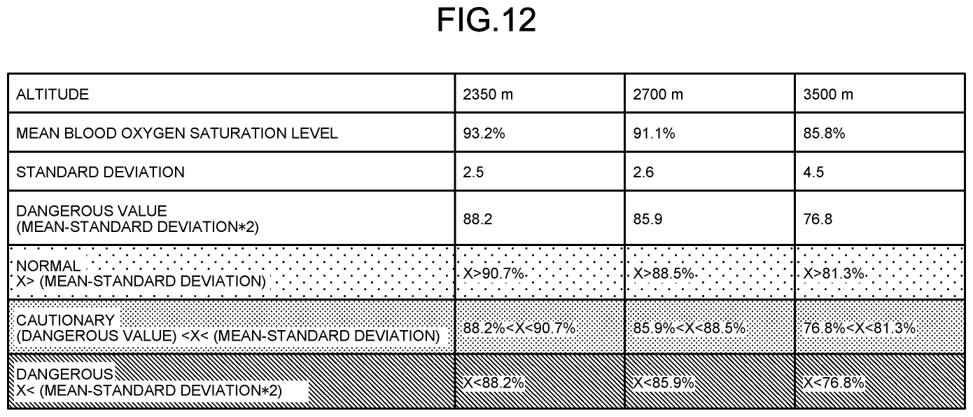

The following describes examples of the determination thresholds of the first device 1100. FIG. 11 is a graph for explaining a relation example between the altitude and the blood oxygen saturation level in the mountaineering. FIG. 12 is a table illustrating the examples of the determination thresholds of the first device 1100.

During the mountaineering, as the altitude increases, the human blood oxygen saturation level decreases, and oxygen in the body also decreases as illustrated in FIG. 11. A mountaineer develops “altitude sickness” due to the lack of oxygen. Although the leader 2100 of the mountaineering tour can recognize the physical conditions of the members 2200 to some extent from facial complexions or behavior of the members 2200, the actual physical conditions can be hard to be recognized by the leader 2100 in detail. For this reason, the sensor system 1000 assists the leader 2100 to quickly recognize the state of ataxia of the member 2200.

The sensor system 1000 has a function to calculate a dangerous value from the mean value and the allowable deviation value of the blood oxygen saturation level for each of a plurality of altitudes, divide a range from the normal level to the dangerous level into a plurality of levels, and create a table indicating a range of allowable values for each of the altitudes. As illustrated in FIG. 12, the sensor system 1000 calculates the mean value of the blood oxygen saturation level for each of the altitudes and applies the mean value to a formula to calculate the standard deviation. The sensor system 1000 calculates the dangerous value for each of the altitudes by subtracting the standard deviation * 2 from the mean value of the blood oxygen saturation level. In FIG. 12, X represents the actual value of the blood oxygen saturation level of the member 2200. For simplicity of explanation, FIG. 12 explains a case of three altitudes of 2350 m, 2700 m, and 3500 m, but the number of altitudes is not limited to this case.

For example, when the sensor system 1000 determines the physical condition of the member 2200 at three levels of normal, cautionary, and dangerous levels, the determination information 151 can be set as described below. The sensor system 1000 sets a normal threshold for determining as normal for each of the altitudes by subtracting the standard deviation from the mean value for each of the altitudes. As a condition to determine the blood oxygen saturation level as cautionary for each of the altitudes, the sensor system 1000 sets the upper limit as the normal threshold and the lower limit as the dangerous value. As a condition to determine the blood oxygen saturation level as dangerous for each of the altitudes, the sensor system 1000 sets the condition that the blood oxygen saturation level is lower than the dangerous value.

In the example illustrated in FIG. 12, the determination information 151 is set as follows: when the altitude is 2350 m, the determination condition for the normal level is set as X>90.7%; the determination condition for the cautionary level is set as 88.2%<X<90.7%; and the determination condition for the dangerous level is set as X<88.2%. The determination information 151 is set as follows: when the altitude is 2700 m, the determination condition for the normal level is set as X>88.5%; the determination condition for the cautionary level is set as 85.9%<X<88.5%; and the determination condition for the dangerous level is set as X<85.9%. The determination information 151 is set as follows: when the altitude is 3500 m, the determination condition for the normal level is set as X>81.3%; the determination condition for the cautionary level is set as 76.8%<X<81.3%; and the determination condition for the dangerous level is set as X<76.8%. As a result, the sensor system 1000 can determine changes in the physical conditions of the members 2200 and notify the leader 2100 of the determination results by comparing the thresholds (determination thresholds) of the determination condition corresponding to the altitudes of the members 2200 with the actual blood oxygen saturation levels.

In the present embodiment, a case will be described where, in the determination information 151, the thresholds of the determination conditions are the determination thresholds, and the determination conditions for the normal, cautionary, and dangerous levels include the respective determination thresholds, but the present disclosure is not limited to this case. For example, the determination information 151 may have a configuration including only a determination threshold that allows determination of whether the level is at the dangerous level. By setting the determination threshold as a threshold of a determination condition for determining that the blood oxygen saturation level is at the dangerous level, for example, the leader 2100 can be warned of the presence of the member 2200 whose blood oxygen saturation level is at the dangerous level at the detected altitude. By setting the determination threshold as a threshold of a determination condition for determining that the blood oxygen saturation level is at the cautionary level, for example, the leader 2100 can be warned of the presence of the member 2200 whose blood oxygen saturation level is at the cautionary level, that is, may change to the dangerous level at the detected altitude. The determination thresholds and the determination conditions may be obtained using, for example, machine learning, and set in the determination information 151.

### 1-5. Display Example of Status Information

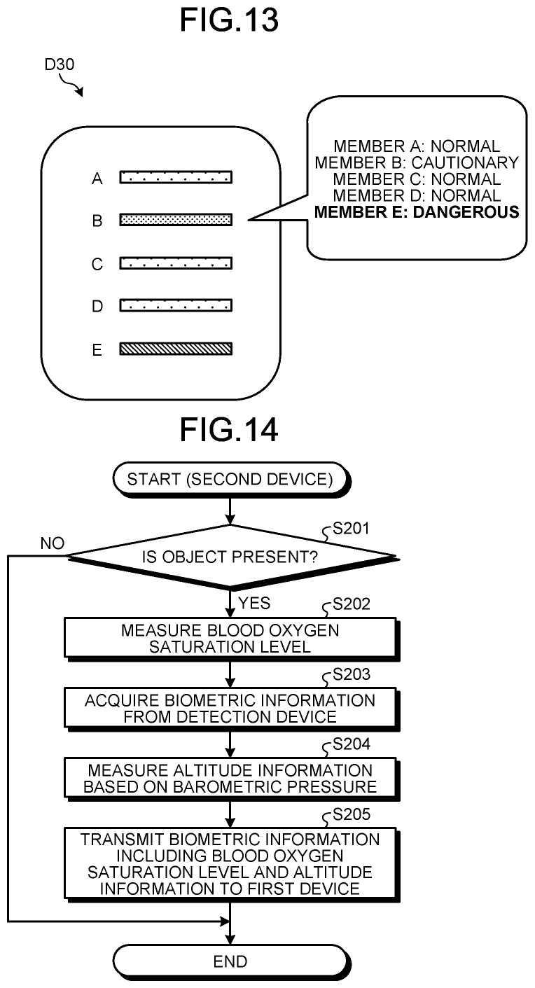

The following describes an example of the status information D30 displayed by the first device 1100. FIG. 13 is a plan view illustrating a display example of the status information D30. As illustrated in FIG. 13, the status information D30 is information indicating the members 2200 and the status of the blood oxygen saturation levels. The status information D30 is information allowing determination of whether the status of each of the blood oxygen saturation levels of five of the members 2200 A, B, C, D, and E is normal, cautionary, or dangerous. In this case, the group information 152 includes the information indicating the relation between each of the five members 2200 A, B, C, D, and E and the second device 1200 set as a slave.

In the example illustrated in FIG. 13, the status information D30 indicates that the status (physical condition) of the members 2200 A, C, and D is normal, the status of the member 2200 B is cautionary, and the status of the member 2200 E is dangerous. In the example illustrated in FIG. 13, A, B, C, D, and E represent the names of the members 2200, but they may be, for example, identification numbers of the members 2200, or identification numbers of the second devices 1200. The status information D30 is configured to indicate the normal status in green, the cautionary status in yellow, and the dangerous status in red. The first device 1100 displays the status information D30 on the display panel 130 in different display appearances for the normal, cautionary, and dangerous states so as to facilitate the leader 2100 to recognize the status of the members 2200.

In the present embodiment, in the sensor system 1000, the first device 1100 acquires the biometric information D10 and the altitude information D20 from the second devices 1200, and determines the changes in the physical conditions of the members 2200 carrying the second devices 1200. In the sensor system 1000, the first device 1100 displays the status information D30 on the display panel 130 so as to allow the leader 2100 to recognize the status of the blood oxygen saturation levels of the members 2200 at a glance. In the example illustrated in FIG. 13, the sensor system 1000 can quickly determine that “member E” is in the dangerous status and “member B” is in the cautionary status among the members 2200, and therefore, can quickly respond to the changes in physical conditions. The first device 1100 may be configured to display only the status information D30 of the members 2200 whose blood oxygen saturation levels are determined to be at least one of the dangerous and cautionary statuses.

### 1-6. Processing Procedure Example of Second Device According to First Embodiment

The following describes a processing procedure of the second device 1200 carried by the member 2200. FIG. 14 is a flowchart illustrating an example of the processing procedure performed by the second device 1200 according to the first embodiment. The processing procedure illustrated in FIG. 14 is implemented by execution of a computer program by the controller 240 of the second device 1200. The second device 1200 executes the processing procedure illustrated in FIG. 14, for example, when a set determination time is reached.

As illustrated in FIG. 14, the second device 1200 determines whether an object is present (Step S201). For example, the second device 1200 determines that the object is present if the biometric information D10 is being able to be measured by the detection device 1, and determines that no object is present if the biometric information D10 is not being able to be measured. If the second device 1200 determines that no object is present (No at Step S201), the processing procedure illustrated in FIG. 14 ends. If the second device 1200 determines that the object is present (Yes at Step S201), processing at Step S202 is performed.

The second device 1200 measures the blood oxygen saturation level (Step S202). For example, the second device 1200 causes the detection device 1 to measure, for example, the blood oxygen saturation level and the pulsation of the living body in the finger Fg or the like of the member 2200, and generates the biometric information D10 indicating the measurement results. After the processing at Step S202 ends, the second device 1200 performs the processing at Step S203.

The second device 1200 acquires the biometric information D10 from the detection device 1 (Step S203). For example, the second device 1200 acquires the biometric information D10 including, for example, the blood oxygen saturation level and the pulsation measured by the detection device 1, and stores the biometric information D10 in the storage 230 in chronological order. After the processing at Step S203 ends, the second device 1200 performs the processing at Step S204.

The second device 1200 measures the altitude information D20 based on the barometric pressure (Step S204). For example, the second device 1200 measures the barometric pressure using the altitude sensor 210, and calculates the altitude of the second device 1200 based on the barometric pressure so as to measure the altitude information D20 indicating, for example, the barometric pressure and the altitude. After the processing at Step S204 ends, the second device 1200 performs the processing at Step S205.

The second device 1200 transmits the biometric information D10 including the blood oxygen saturation level and the altitude information D20 to the first device 1100 (Step S205). For example, the second device 1200 transmits the biometric information D10 and the altitude information D20 to the first device 1100 serving as the master indicated by the setting information 231 through the communicator 220. The second device 1200 transmits the biometric information D10 and the altitude information D20 to the first device 1100 in association with information allowing identification of the second device 1200 and the member 2200 carrying the second device 1200. After the processing at Step S205 ends, the second device 1200 ends the processing procedure illustrated in FIG. 14.

### 1-7. Processing Procedure Example of First Device According to First Embodiment

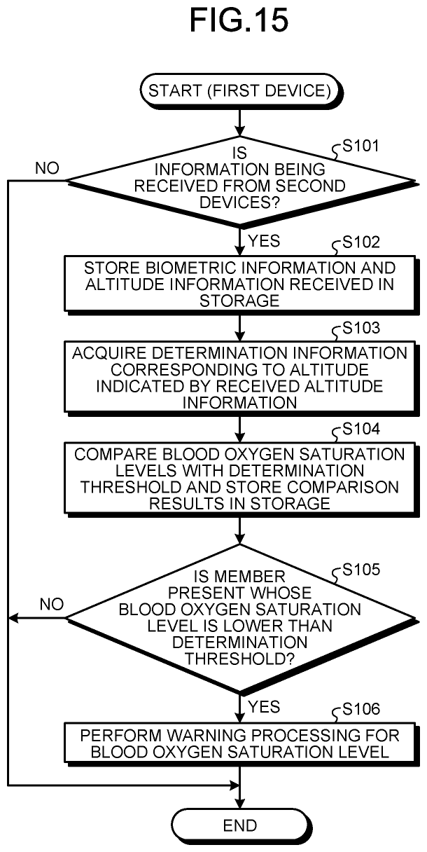

The following describes a processing procedure of the first device 1100 used by the leader 2100. FIG. 15 is a flowchart illustrating an example of the processing procedure performed by the first device 1100 according to the first embodiment. The processing procedure illustrated in FIG. 15 is implemented by execution of a computer program by the controller 160 of the first device 1100. The first device 1100, for example, repeatedly executes the processing procedure illustrated in FIG. 15 during a target period for assisting the leader 2100.

As illustrated in FIG. 15, the first device 1100 determines whether information is being received from the second devices 1200 (Step S101). For example, if the first device 1100 is receiving, through the communicator 120, information from the second devices 1200 serving as the slaves indicated by the group information 152, the first device 1100 determines that information is being received from the second devices 1200. If the first device 1100 determines that no information is being received from the second devices 1200 (No at Step S101), the processing procedure illustrated in FIG. 15 ends. If the first device 1100 determines that information is being received from the second devices 1200 (Yes at S101), processing at Step S102 is performed.

The first device 1100 stores the received biometric information D10 and the received altitude information D20 in the storage 150 (Step S102). For example, the first device 1100 stores the biometric information D10 and the altitude information D20 received from the second devices 1200 in the storage 150 in association with the second devices 1200 and the members 2200 carrying the second devices 1200. After the processing at Step S102 ends, the first device 1100 performs processing at Step S103.

The first device 1100 acquires the determination information 151 corresponding to the altitude indicated by the received altitude information D20 (Step S103). For example, if the altitude information D20 indicates 2350 m, the first device 1100 acquires the determination information 151 including the determination threshold corresponding to 2350 m illustrated in FIG. 12 from the storage 150. After the processing at Step S103 ends, the first device 1100 performs processing at Step S104.

The first device 1100 compares the blood oxygen saturation levels with the determination threshold and stores the comparison results in the storage 150 (Step S104). For example, the first device 1100 stores the comparison results of whether each of the blood oxygen saturation levels is lower than the determination threshold indicating the dangerous status in the determination information 151 in the storage 150 in association with the member 2200. In the present embodiment, if the blood oxygen saturation level is not lower than the determination threshold indicating the dangerous status in the determination information 151, the first device 1100 determines whether the blood oxygen saturation level meets the determination condition of the normal or cautionary status, and stores the determination results in the storage 150. Thus, the first device 1100 stores, in the storage 150, the information allowing determination of whether the status of the blood oxygen saturation level of each of the members 2200 is normal, cautionary, or dangerous. After the processing at Step S104 ends, the first device 1100 performs processing at Step S105.

The first device 1100 determines whether the member 2200 is present whose blood oxygen saturation level is lower than the determination threshold (Step S105). For example, if the latest determination results of the members 2200 stored as management targets in the storage 150 include a determination result that the blood oxygen saturation level is lower than the determination threshold for the dangerous status, the first device 1100 determines that the member 2200 is present whose blood oxygen saturation level is lower than the determination threshold because the member 2200 with an abnormal blood oxygen saturation level is present. For example, if the latest determination results of the members 2200 stored as the management targets in the storage 150 include a determination result that the blood oxygen saturation level is lower than the determination threshold (upper limit value) for the cautionary status, the first device 1100 may determine that the member 2200 is present whose blood oxygen saturation level is lower than the determination threshold because the member 2200 with an abnormal or cautionary blood oxygen saturation level is present. If the first device 1100 determines that none of the members 2200 is present whose blood oxygen saturation level is lower than the determination threshold (No at Step S105), the processing procedure illustrated in FIG. 15 ends. If the first device 1100 determines that the member 2200 is present whose blood oxygen saturation level is lower than the determination threshold (Yes at Step S105), processing at Step S106 is performed.

The first device 1100 performs warning processing for the blood oxygen saturation level (Step S106). The warning processing includes, for example: processing to warn that the blood oxygen saturation level of the member 2200 is in a warning status, using display, sound and the like; processing to output an alarm; processing to notify of the member 2200 with the abnormal blood oxygen saturation level; and processing to display the status information D30 indicating the state of the blood oxygen saturation level of each of the members 2200. The first device 1100 performs the warning processing to assist the leader 2100 to recognize the member 2200 with the worsened blood oxygen saturation level. After the processing at Step S106 ends, the first device 1100 ends the processing procedure illustrated in FIG. 15.

In the processing procedure illustrated in FIG. 15, when the first device 1100 determines that no member 2200 is present whose blood oxygen saturation level is lower than the determination threshold, the first device 1100 ends the processing procedure, but may perform processing to, for example, display the latest status information D30 on the display panel 130.

As described above, in the sensor system 1000, the second device 1200 of each of the members 2200 transmits the biometric information D10 and the altitude information D20 to the first device 1100. The sensor system 1000 warns of the member 2200 whose blood oxygen level is lower than the determination threshold corresponding to the altitude among the members 2200 based on the biometric information D10 and the altitude information D20 received by the first device 1100 from the second devices 1200. Thus, the first device 1100 warns of the dangerous blood oxygen saturation level, so that the sensor system 1000 can cause the leader 2100 to easily recognize a change in physical conditions of the members 2200 to be managed. As a result, the sensor system 1000 can warn the leader 2100 that the physical condition of the member 2200 belonging to the group has worsened so as to be capable of supporting a quick response to the change in the physical condition. In particular, during the mountaineering, even if the leader 2100 cannot keep track of the facial complexions or behavior of the members 2200, the sensor system 1000 can reduce the risk of occurrence of the altitude sickness by causing the leader 2100 to recognize the member 2200 whose physical condition has worsened.

In the sensor system 1000, the first device 1100 displays the status information D30 allowing determination of whether the blood oxygen saturation level measured by each of the second devices 1200 is in the dangerous status on the display panel 130. Thus, the first device 1100 displays the status information D30 on the display panel 130, so that the sensor system 1000 can cause the leader 2100 to quickly recognize whether the blood oxygen saturation level of each of the members 2200 is in the dangerous status. As a result, even if the leader 2100 cannot view the facial complexions or behavior of all the members 2200, the sensor system 1000 can cause the leader 2100 to recognize the physical conditions of the members 2200. The first device 1100 uses the status information D30 to display stepwise the normal, cautionary, or dangerous status of the blood oxygen saturation level, so that the sensor system 1000 can cause the leader 2100 to quickly recognize the change in the blood oxygen saturation level of each of the members 2200.

In addition, the first device 1100 determines the blood oxygen saturation level using the determination threshold corresponding to the altitude, so that the sensor system 1000 can determine the blood oxygen saturation level suitable for each altitude, even if the members 2200 are located at different altitudes. For example, at a construction site, even if the members 2200 are working on different floors in the same building, the sensor system 1000 can determine the blood oxygen levels of all the members 2200. Therefore, the sensor system 1000 can determine the blood oxygen saturation levels of the members 2200 located over a wide range of different altitudes, and thereby can improve the convenience of the system.

### 2. Modification of First Embodiment

In the first embodiment described above, the case has been described where, in the sensor system 1000, the first device 1100 illustrated in FIG. 10 does not measure the status of the leader 2100. However, the present disclosure is not limited to this case, and the first device 1100 can be replaced with a first device 1100A to be described below. That is, the sensor system 1000 may have a configuration including the first device 1100A and the second devices 1200. The first device 1100A is a device wearable by the leader 2100 in the same manner as the second device 1200 described above. In the following description, the same components as those of the first embodiment are denoted by the same reference numerals and will not be described.

### 2-1 First Device According to Modification of First Embodiment

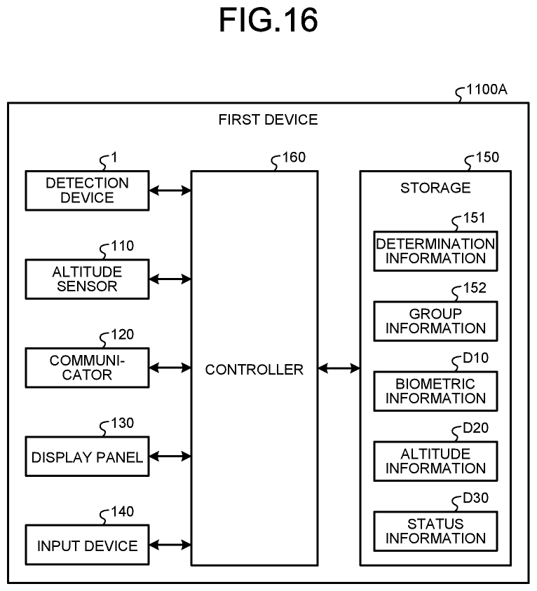

The following describes a functional configuration of the first device 1100A according to a modification of the first embodiment. FIG. 16 is a configuration diagram illustrating an example of the functional configuration of the first device 1100A according to the modification of the first embodiment. As illustrated in FIG. 16, the first device 1100A includes the detection device 1 described above, the altitude sensor 110, the communicator 120, the display panel 130, the input device 140, the storage 150, and the controller 160. The controller 160 is electrically coupled to the detection device 1, the altitude sensor 110, the communicator 120, the display panel 130, the input device 140, and the storage 150. The first device 1100A may be implemented, for example, as a combination of a finger ring or a wristband and a smartphone, or as a smartwatch.

The detection device 1 detects the biometric information D10 on the living body in the finger Fg or the like of the leader 2100, and supplies the biometric information D10 to the controller 160. The detection device 1 supplies the biometric information D10 to the controller 160, for example, each time the biometric information D10 is measured at predetermined timing. In the present embodiment, the detection device 1 serves as the blood oxygen saturation level sensor by supplying the biometric information including the blood oxygen saturation level to the controller 160.

The controller 160 stores the biometric information D10 supplied by the detection device 1 as information on the leader 2100 in the storage 150. The controller 160 has a function to manage the measurement results measured by the detection devices 1 of the first device 1100A and the second devices 1200, and warn that the blood oxygen saturation level is lower than the determination threshold corresponding to the altitude information D20. The controller 160 can warn of the abnormality in the blood oxygen saturation level of each of the leader 2100 and the members 2200, and thereby can also assist the leader 2100 in performing the physical condition management. The controller 160 has a function to display the status information D30 allowing determination of whether the blood oxygen saturation level measured by each of the first device 1100A and the second devices 1200 is in the dangerous range on the display panel 130.

The above has described the functional configuration example of the first device 1100A according to the modification of the first embodiment. The configuration described above using FIG. 16 is merely an example. The functional configuration of the first device 1100A according to the present modification of the first embodiment is not limited to this example. The functional configuration of the first device 1100A according to the present modification of the first embodiment can be flexibly modified according to specifications and operations.

### 2-2. Processing Procedure Example of First Device According to Modification of First Embodiment

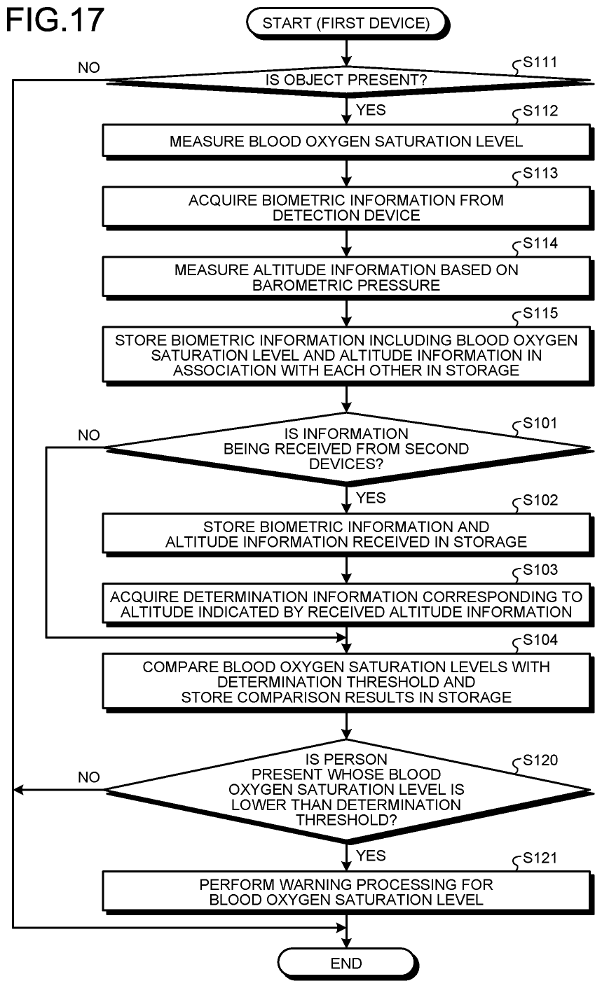

The following describes a processing procedure of the first device 1100A used by the leader 2100. FIG. 17 is a flowchart illustrating an example of the processing procedure performed by the first device 1100A according to the modification of the first embodiment. The processing procedure illustrated in FIG. 17 is implemented by execution of a computer program by the controller 160 of the first device 1100A. The first device 1100A, for example, repeatedly executes the processing procedure illustrated in FIG. 17 during the target period for assisting the leader 2100.

As illustrated in FIG. 17, the first device 1100A determines whether an object is present (Step S111). For example, the first device 1100A determines that the object is present if the biometric information D10 is being able to be measured by the detection device 1, and determines that no object is present if the biometric information D10 is not being able to be measured. If the first device 1100A determines that no object is present (No at Step S111), the processing procedure illustrated in FIG. 17 ends. If the first device 1100A determines that the object is present (Yes at Step S111), processing at Step S112 is performed.

The first device 1100A measures the blood oxygen saturation level (Step S112). For example, the first device 1100A causes the detection device 1 to measure, for example, the blood oxygen saturation level and the pulsation of the living body of the leader 2100, and generates the biometric information D10 representing the measurement results. After the processing at Step S112 ends, the first device 1100A performs the processing at Step S113.

The first device 1100A acquires the biometric information D10 from the detection device 1 (Step S113). For example, the first device 1100A acquires the biometric information D10 including, for example, the blood oxygen saturation level and the pulsation measured by the detection device 1, and stores the biometric information D10 in the storage 150. After the processing at Step S113 ends, the first device 1100A performs the processing at Step S114.

The first device 1100A measures the altitude information D20 based on the barometric pressure (Step S114). For example, the first device 1100A measures the barometric pressure using the altitude sensor 110, and calculates the altitude of the first device 1100A based on the barometric pressure so as to measure the altitude information D20 indicating, for example, the barometric pressure and the altitude. After the processing at Step S114 ends, the first device 1100A performs the processing at Step S115.

The first device 1100A stores the biometric information D10 including the blood oxygen saturation level and the altitude information D20 in association with each other in the storage 150 (Step S115). For example, the first device 1100A stores the biometric information D10 and the altitude information D20 in the storage 150 in association with information identifiable by the leader 2100. After the processing at Step S115 ends, the first device 1100A performs the processing at Step S101.

The processing procedure through the following Steps S101 to S104 is the same as that through Steps S101 to S104 illustrated in FIG. 15, and therefore, will be briefly described.

The first device 1100A determines whether information is being received from the second devices 1200 (Step S101). If the first device 1100A determines that no information is being received from the second devices 1200 (No at Step S101), the processing at Step S104 is performed. The first device 1100A compares the blood oxygen saturation levels with the determination threshold and stores the comparison results in the storage 150 (Step S104). That is, the first device 1100A compares the blood oxygen saturation level of the leader 2100 acquired by the first device 1100A from the detection device 1 with the determination threshold, and stores the comparison result in the storage 150. After the processing at Step S104 ends, the first device 1100A performs processing at Step S120 to be described later.

If the first device 1100A determines that information is being received from the second devices 1200 (Yes at S101), the processing at Step S102 is performed. The first device 1100A stores the received biometric information D10 and the received altitude information D20 in the storage 150 (Step S102). For example, the first device 1100A stores the biometric information D10 and the altitude information D20 received from the second devices 1200 in the storage 150 in association with the second devices 1200 and the members 2200 carrying the second devices 1200. After the processing at Step S102 ends, the first device 1100A performs the processing at Step S103.

The first device 1100A acquires the determination information 151 corresponding to the altitude indicated by the received altitude information D20 (Step S103). The first device 1100A then compares the blood oxygen saturation levels with the determination threshold and stores the comparison results in the storage 150 (Step S104). That is, the first device 1100A compares the blood oxygen saturation level of the leader 2100 acquired by the first device 1100A from the detection device 1 and the blood oxygen saturation levels of the members 2200 from the second devices 1200 with the determination threshold, and stores the comparison results in the storage 150. After the processing at Step S104 ends, the first device 1100A performs the processing at Step S120.

The first device 1100A determines whether a person is present whose blood oxygen saturation level is lower than the determination threshold (Step S120). For example, if the latest determination results of the leader 2100 and the members 2200 as management targets stored in the storage 150 include a determination result that the blood oxygen saturation level is lower than the determination threshold for the dangerous status, the first device 1100A determines that a person is present whose blood oxygen saturation level is lower than the determination threshold because the person with an abnormal blood oxygen saturation level is present. For example, if the latest determination results of the leader 2100 and the members 2200 as management targets stored in the storage 150 include a determination result that the blood oxygen saturation level is lower than the determination threshold (upper limit value) for the cautionary status, the first device 1100A may determine that a person is present whose blood oxygen saturation level is lower than the determination threshold because the person with an abnormal or cautionary blood oxygen saturation level is present. If the first device 1100A determines that no person is present whose blood oxygen saturation level is lower than the determination threshold (No at Step S120), the processing procedure illustrated in FIG. 17 ends. If the first device 1100A determines that a person is present whose blood oxygen saturation level is lower than the determination threshold (Yes at Step S120), processing at Step S121 is performed.

The first device 1100A performs warning processing for the blood oxygen saturation level (Step S121). The warning processing includes, for example: processing to warn that the blood oxygen saturation level of at least one of the leader 2100 and the members 2200 is in a warning status, using display, sound or the like; processing to output an alarm; processing to notify of the person with the abnormal blood oxygen saturation level; and processing to display the status information D30 indicating the state of the blood oxygen saturation level of each of the leader 2100 and the members 2200. The first device 1100A performs the warning processing to assist the leader 2100 to recognize the leader 2100 or the member 2200 with the worsened blood oxygen saturation level. After the processing at Step S121 ends, the first device 1100A ends the processing procedure illustrated in FIG. 17.

As described above, in the sensor system 1000, the second device 1200 of each of the members 2200 transmits the biometric information D10 and the altitude information D20 to the first device 1100A, and the first device 1100A warns that the blood oxygen level of the member 2200 is lower than the determination threshold corresponding to the altitude. Furthermore, the sensor system 1000 acquires the biometric information D10 and the altitude information D20 about the leader 2100, and warns that the blood oxygen level is lower than the determination threshold corresponding to the altitude. Thus, the first device 1100A warns of the dangerous blood oxygen saturation level, so that the sensor system 1000 can cause the leader 2100 to easily recognize a change in physical conditions of the leader 2100 and the members 2200. As a result, the sensor system 1000 can warn the leader 2100 that the physical condition of the leader 2100 or the member 2200 has worsened so as to be capable of supporting a quick response to the change in the physical condition that occurs in the group.

### 3. Second Embodiment

### 3-1. Sensor System According to Second Embodiment

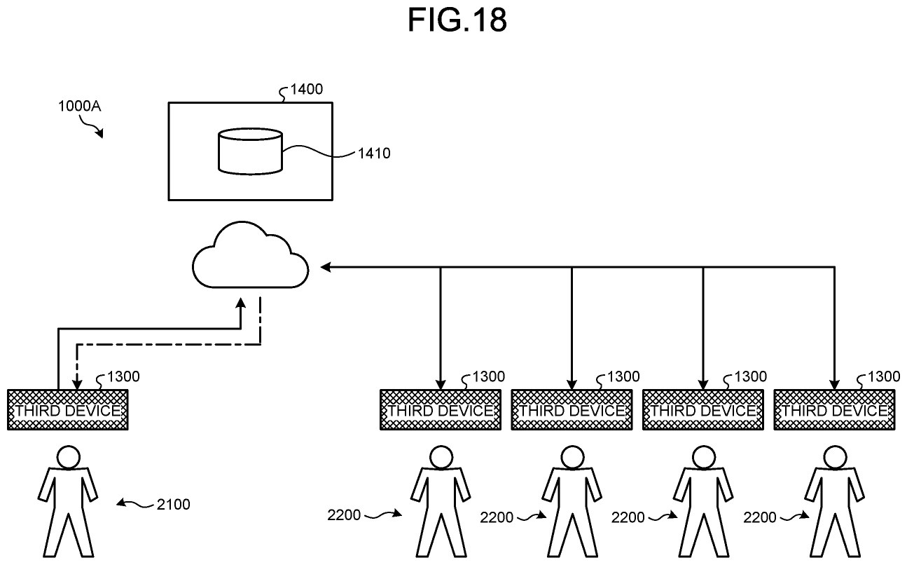

FIG. 18 is a configuration diagram illustrating an exemplary system configuration of a sensor system according to a second embodiment. As illustrated in FIG. 18, a sensor system 1000A can provide a function capable of keeping track of a change in physical conditions of the members 2200 in the group to which the leader 2100 and the members 2200 belong. The sensor system 1000A includes a plurality of third devices 1300. The third devices 1300 are wearable devices, such as wristbands, finger rings, or smartwatches, that can be carried by the leader 2100 and the members 2200. Each of the third devices 1300 is configured to be capable of accessing a server 1400 in a cloud. The server 1400 is a network server device, and includes a data storage 1410. The data storage 1410 stores therein information indicating determination results of the biometric information D10 measured by the third devices 1300, and shares the stored information with the leader 2100 and the members 2200.

### 3-2. Third Device

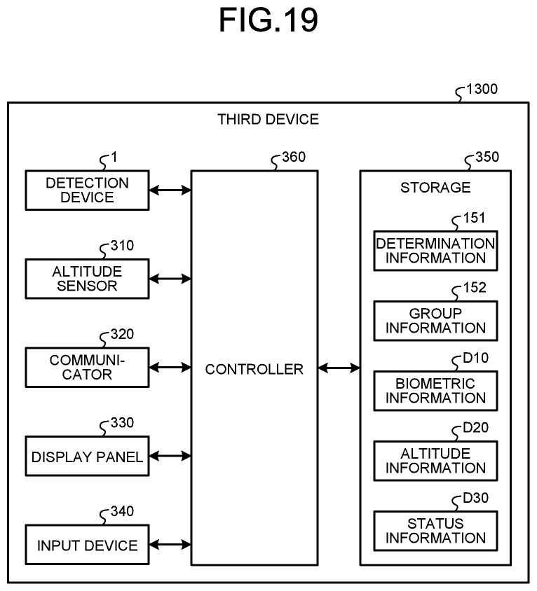

The following describes a functional configuration of the third device 1300. FIG. 19 is a configuration diagram illustrating an example of the functional configuration of the third device 1300 according to the second embodiment. As illustrated in FIG. 19, the third device 1300 includes the detection device 1 described above, an altitude sensor 310, a communicator (communication circuit) 320, a display panel 330, an input device 340, a storage (storage circuit) 350, and a controller (control circuit) 360. The controller 360 is electrically coupled to the detection device 1, the altitude sensor 310, the communicator 320, the display panel 330, the input device 340, and the storage 350.

The detection device 1 detects the biometric information D10 on the living body of the leader 2100 or the member 2200 and supplies the biometric information D10 to the controller 360. The detection device 1 supplies the biometric information D10 to the controller 360, for example, each time the biometric information D10 is measured at predetermined timing. In the present embodiment, the detection device 1 serves as the blood oxygen saturation level sensor by supplying the biometric information including the blood oxygen saturation level to the controller 360.

The altitude sensor 310 includes, for example, a barometric pressure sensor and a GPS receiver. The altitude sensor 310 has a function to detect the ambient barometric pressure acting on a third device 1300 and convert the barometric pressure into the altitude. The altitude sensor 310 calculates the altitude (elevation) of the current position (latitude and longitude) of the third device 1300 measured by the GPS receiver, based on the relation of the latitude and the longitude with the height indicated by the map information. The altitude sensor 310 supplies the altitude information D20 indicating, for example, the altitude and the barometric pressure detected to the controller 360.

The communicator 320 wirelessly communicates. The communicator 320 supports the wireless communication standards. Examples of the communication standards include the cellular phone communication standards, such as the 3G, the 4G, and the 5G standards, and the short-range wireless communication standards. The communicator 320 supplies received information to the controller 360. The communicator 320 transmits various types of information requested by the controller 360 to destinations, such as the server 1400 and the other third devices 1300.

The display panel 330 has a function to display various types of information. The display panel 330 displays, for example, information received from the server 1400 and information for assisting. The controller 360 controls display of the display panel 330. For example, the display device that displays various types of information can be used as the display panel 330. Examples of the display device include the liquid crystal display and the organic EL display.

The input device 340 has a function to detect physical input operations by the user. The input device 340 includes operation devices, such as the touchscreen and operation buttons. The input device 340 supplies input information representing the detected input operations to the controller 360.

The storage 350 stores therein the computer programs and data. The storage 350 temporarily stores therein the processing results of the controller 360. The storage 350 includes the storage media. The storage media include, for example, the ROM, the RAM, the memory card, the optical disc, and/or the magneto-optical disk. The storage 350 stores therein information indicating, for example, detection results detected by the altitude sensor 310. The storage 350 stores therein various types of information, such as the determination information 151, the group information 152, the biometric information D10, the altitude information D20, and the status information D30 described above.

The controller 360 includes, for example, the MCU and the CPU. The controller 360 integrally controls the operation of the third device 1300. The controller 360 has, for example, a function to acquire information received from, for example, the servers 1400 and the other third devices 1300 through the communicator 320. The controller 360 has a function to share the status information D30 allowing determination of whether the blood oxygen saturation level measured by the detection device 1 is lower than the determination threshold corresponding to the altitude information D20, among the third devices 1300. The controller 360 has a function of acquire the status information D30 shared by the other third devices 1300 and display it on the display panel 330. The status information D30 indicates stepwise the normal, cautionary, or dangerous status of the blood oxygen saturation level of each of the leader 2100 and the members 2200. The controller 360 can display the status information D30 on the display panel 330 at all times or at a warning time. Various functions of the controller 360 are performed by executing the computer programs.

The above has described the functional configuration example of the third device 1300 according to the present embodiment. The configuration described above using FIG. 19 is merely an example. The functional configuration of the third device 1300 according to the present embodiment is not limited to this example. The functional configuration of the third device 1300 according to the present embodiment can be flexibly modified according to specifications and operations.

### 3-3. Processing Procedure Example of Third Device According to Second Embodiment

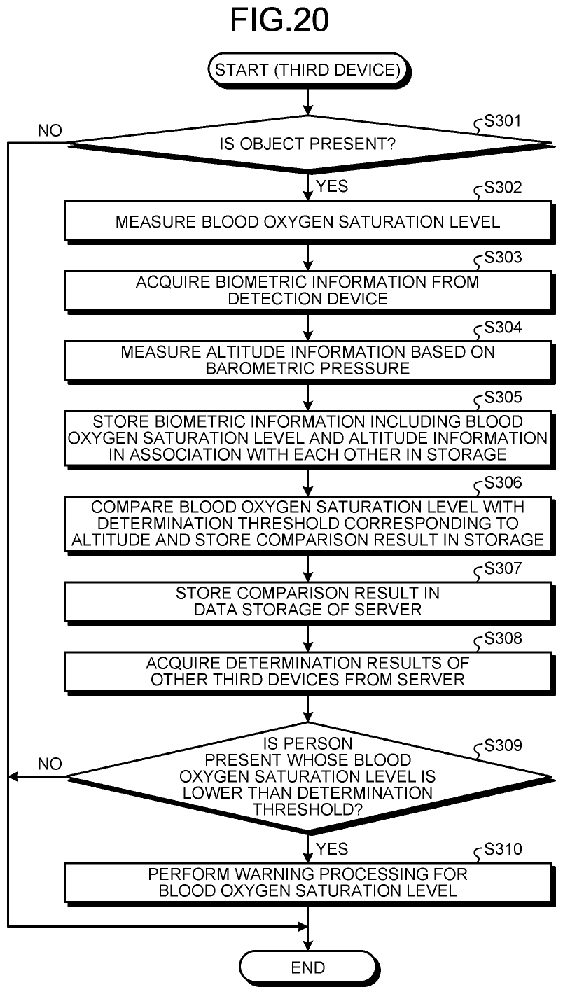

The following describes a processing procedure of each of the third devices 1300 used by the leader 2100 and the members 2200. FIG. 20 is a flowchart illustrating an example of the processing procedure performed by the third device 1300 according to the second embodiment. The processing procedure illustrated in FIG. 20 is implemented by execution of a computer program by the controller 360 of the third device 1300. The third device 1300, for example, repeatedly executes the processing procedure illustrated in FIG. 20 during the target period for assisting the leader 2100.

As illustrated in FIG. 20, the third device 1300 determines whether an object is present (Step S301). For example, the third device 1300 determines that the object is present if the biometric information D10 is being able to be measured by the detection device 1, and determines that no object is present if the biometric information D10 is not being able to be measured. If the third device 1300 determines that no object is present (No at Step S301), the processing procedure illustrated in FIG. 20 ends. If the third device 1300 determines that the object is present (Yes at Step S301), processing at Step S302 is performed.

The third device 1300 measures the blood oxygen saturation level (Step S302). For example, the third device 1300 causes the detection device 1 to measure, for example, the blood oxygen saturation level and the pulsation of the living body of the leader 2100 or the member 2200, and generates the biometric information D10 representing the measurement results. After the processing at Step S302 ends, the third device 1300 performs processing at Step S303.

The third device 1300 acquires the biometric information D10 from the detection device 1 (Step S303). For example, the third device 1300 acquires the biometric information D10 including, for example, the blood oxygen saturation level and the pulsation measured by the detection device 1, and stores the biometric information D10 in the storage 350. After the processing at Step S303 ends, the third device 1300 performs processing at Step S304.

The third device 1300 measures the altitude information D20 based on the barometric pressure (Step S304). For example, the third device 1300 measures the barometric pressure using the altitude sensor 110, and calculates the altitude of the third device 1300 based on the barometric pressure so as to measure the altitude information D20 indicating, for example, the barometric pressure and the altitude. After the processing at Step S304 ends, the third device 1300 performs processing at Step S305.

The third device 1300 stores the biometric information D10 including the blood oxygen saturation level and the altitude information D20 in association with each other in the storage 350 (Step S305). For example, the third device 1300 stores the biometric information D10 and the altitude information D20 in association with each other in the storage 350. After the processing at Step S305 ends, the third device 1300 performs processing at Step S306.

The third device 1300 compares the blood oxygen saturation level with the determination threshold corresponding to the altitude, and stores the comparison result in the storage 350 (Step S306). For example, the third device 1300 acquires the determination information 151 corresponding to the altitude indicated by the altitude information D20, and stores the comparison result obtained by comparing the determination threshold indicated by the determination information 151 with the blood oxygen saturation level in the storage 350. In the present embodiment, the comparison result indicates, for example, whether the blood oxygen saturation level is at the normal, cautionary, or warning level described above. After the processing at Step S306 ends, the third device 1300 performs processing at Step S307.

The third device 1300 stores the comparison result in the data storage (storage circuit) 1410 of the server 1400 (Step S307). For example, the third device 1300 requests the server 1400 to save the comparison result of Step S306 through the communicator 320. This request causes the server 1400 to store the information indicating the determination result of the blood oxygen saturation level in the data storage 1410 so as to allow identification of the third device 1300 and the group. After the processing at Step S307 ends, the third device 1300 performs processing at Step S308.

The third device 1300 acquires the determination results of the other third devices 1300 from the server 1400 (Step S308). For example, the third device 1300 requests the server 1400 through the communicator 320 to acquire the determination results of the third devices 1300 belonging to the group indicated by the group information 152. This request causes the server 1400 to extract the requested determination results of the group from the data storage 1410 and supply the determination results to the requesting third device 1300. The third device 1300 shares the status information D30 among the third devices 1300 by storing the determination results acquired from the server 1400 in the status information D30 in the storage 350 so as to allow identification of the leader 2100 or the member 2200. Then, the third device 1300 performs processing at Step S309.

The third device 1300 determines whether a person is present whose blood oxygen saturation level is lower than the determination threshold (Step S309). For example, if the comparison results acquired from the server 1400 include a determination result that the blood oxygen saturation level is lower than the determination threshold, the third device 1300 determines that a person is present whose blood oxygen saturation level is lower than the determination threshold. If the third device 1300 determines that no person is present whose blood oxygen saturation level is lower than the determination threshold (No at Step S309), the processing procedure illustrated in FIG. 20 ends. If the third device 1300 determines that a person is present whose blood oxygen saturation level is lower than the determination threshold (Yes at Step S309), processing at Step S310 is performed.

The third device 1300 performs the warning processing for the blood oxygen saturation level (Step S310). The warning processing includes, for example: processing to warn that the blood oxygen saturation level of each of the leader 2100 and the members 2200 is in a warning status, using display, sound and the like; processing to output an alarm; processing to notify of, for example, the leader 2100 or the member 2200 with the abnormal blood oxygen saturation level; and processing to display the status information D30 indicating the state of the blood oxygen saturation level of each of the leader 2100 and the members 2200. The third device 1300 performs the warning processing to assist the leader 2100 to recognize, for example, the leader 2100 or the member 2200 with the worsened blood oxygen saturation level. After the processing at Step S310 ends, the third device 1300 ends the processing procedure illustrated in FIG. 20.

As described above, in the sensor system 1000A, each of the leader 2100 and the members 2200 carries the third device 1300, and each of the third devices 1300 determines whether the blood oxygen level is lower than the determination threshold corresponding to the altitude. The sensor system 1000A shares the determination result made by each of the third devices 1300 as to whether the blood oxygen level is lower than the determination threshold corresponding to the altitude, among the third devices 1300. Thus, the sensor system 1000A can cause not only the leader 2100 but all the people in the group to know the status of the blood oxygen saturation level of each other. As a result, the leader 2100 and the members 2200 can recognize the blood oxygen saturation level of each other, so that a sensor system 1000A can assist the quick response to the change in the physical condition that occurs in the group.

The components in the embodiments described above can be combined as appropriate. Other operational advantages accruing from the aspects described in the embodiments of the present invention that are obvious from the description herein, or that are conceivable as appropriate by those skilled in the art will naturally be understood as accruing from the present invention.

What is claimed is:

1. A sensor system comprising:
a first device comprising a first controller and a first communicator; and
a plurality of second devices each comprising an altitude sensor, a blood oxygen saturation level sensor, a second controller, and a second communicator, wherein
the second controller of the second device is configured to transmit altitude information measured by the altitude sensor and biometric information measured by the blood oxygen saturation level sensor to the first device through the second communicator,
the first device has determination information comprising determination thresholds for a blood oxygen saturation level corresponding to different altitudes, and
the first controller is configured to warn that the blood oxygen saturation level is lower than the determination threshold corresponding to the altitude information based on the altitude information and the biometric information received from each of the second devices through the first communicator and the determination information.

2. The sensor system according to claim 1, wherein the first device is configured to display status information on a display panel, the status information allowing determination of whether the blood oxygen saturation level measured by each of the second devices is in a dangerous range.

3. The sensor system according to claim 1, wherein
the first device comprises the altitude sensor and the blood oxygen saturation level sensor, and
the first controller is configured to manage measurement results measured by the first device and the second devices, and warn that the blood oxygen saturation level is lower than the determination threshold corresponding to the altitude information.

4. The sensor system according to claim 1, wherein the first device or the second device is a wristband or a finger ring.

5. The sensor system according to claim 1, wherein the first device is configured to set the second devices as communicable slaves.

6. The sensor system according to claim 1, wherein the blood oxygen saturation level sensor comprises a sensor substrate, a sensor disposed along the sensor substrate, and a plurality of light sources arranged so as to correspond to the arrangement of the sensor.

7. The sensor system according to claim 1, wherein the altitude sensor is a barometric pressure sensor.

8. The sensor system according to claim 1, wherein the altitude sensor is configured to calculate the altitude based on the Global Positioning System and map information.

9. A sensor system comprising a plurality of wearable devices each comprising a controller, an altitude sensor, a blood oxygen saturation level sensor, and a communicator, wherein
each of the wearable devices has determination information comprising determination thresholds for a blood oxygen saturation level corresponding to different altitudes, and
the controller is configured to share status information among the wearable devices, the status information allowing determination of whether the blood oxygen saturation level measured by the blood oxygen saturation level sensor is lower than the determination threshold corresponding to altitude information measured by the altitude sensor.

10. The sensor system according to claim 9, wherein the wearable device is configured to acquire the status information shared by the other wearable devices, and display the acquired status information on a display unit.

11. The sensor system according to claim 9, wherein the wearable device is a wristband or a finger ring.

12. The sensor system according to claim 9, wherein the blood oxygen saturation level sensor comprises a sensor substrate, a sensor disposed along the sensor substrate, and a plurality of light sources arranged so as to correspond to the arrangement of the sensor.

13. The sensor system according to claim 9, wherein the altitude sensor is a barometric pressure sensor.

14. The sensor system according to claim 9, wherein the altitude sensor is configured to calculate the altitude based on the Global Positioning System and map information.
